(19) United States
(12) Patent Application Publication
(10) Pub. No.: US 2006/0211059 A1
(43) Pub. Date: Sep. 21, 2006

(54) Methods of improving screening, diagnosis and staging of prostate cancer using serum testosterone

(76) Inventor: Samir S. Taneja, Wyckoff, NJ (US)

Correspondence Address: KLAUBER & JACKSON, 411 HACKENSACK AVENUE, HACKENSACK, NJ, 07601, US

(21) Appl. No.: 11/377,097

(22) Filed: Mar. 16, 2006

Related U.S. Application Data
(60) Provisional application No. 60/662,955, filed on Mar. 18, 2005.

(51) Int. Cl. G01N 33/574; A61B 8/00

(52) U.S. Cl. 435/7.23; 600/443

(57) ABSTRACT

The present invention provides methods of screening for, detecting or diagnosing prostate cancer in a subject by determining the level of prostate specific antigen (PSA), complexed PSA (cPSA), free PSA, B-PSA, PRO-PSA or HK2 in a biological sample from the subject and correcting this level for free/bioavailable serum testosterone, total testosterone or sex hormone binding globulin. The present invention further relates to identifying a subject at risk for developing prostate cancer or for determining the effectiveness of anti-cancer therapy in a subject having prostate cancer, or for detecting cancer recurrence by determining the level of prostate specific antigen (PSA), complexed PSA (cPSA), free PSA, B-PSA, PRO-PSA or HK2 in a biological sample from the subject and correcting this level for free or bioavailable serum testosterone, total testosterone or a testosterone bound protein, including but not limited to sex hormone binding globulin.

### CROSS-REFERENCE TO RELATED APPLICATIONS

The present application is a non-provisional application claiming the priority of copending provisional application Ser. No. 60/662,955, filed Mar. 18, 2005, the disclosure of which is incorporated by reference herein in its entirety. Applicants claim the benefits of this application under 35 U.S.C. §119 (e).

### FIELD OF THE INVENTION

The present invention relates generally to methods of screening, detecting or diagnosing prostate cancer. The present invention further relates to methods for identifying a subject at risk for developing prostate cancer or for determining the effectiveness of anti-cancer therapy in a subject having prostate cancer.

### BACKGROUND

Prostate cancer is the most frequently diagnosed cancer in the United States, with over a quarter of a million of new cases being diagnosed each year. Despite the roughly $4 billion dollars per year spent treating this disease, forty thousand men die every year due to prostate cancer, which makes prostate cancer the second leading cause of cancer death in men. It is generally acknowledged in the medical community that most, if not all men will eventually develop prostate cancer, provided that they live long enough for the condition to develop. For example, 50% of all men over 50, and essentially all men over 70 suffer from some form of prostate hyperplasia.

One characteristic of prostate cancer is that it generally arises relatively late in life and then progresses slowly. If this were always true, the optimal medical response would be to simply monitor the progression of the cancer rather than aggressively treating it, since by the time the cancer progressed to a life threatening stage, the patient would have likely expired due to other more rapidly progressing factors. However, prostate cancers are highly heterogeneous in their progression. Some cancers grow very rapidly and need to be treated aggressively, whereas others are very slow growing and not life-threatening.

Considering the severe side-effects and expense associated with treating cancer, and prostate cancer treatment in particular, better prognosis tools are desperately needed. Therefore, there is a need to identify other markers that are diagnostic of cancer, and prostate cancer in particular. Furthermore, there is a need to identify a means that can be used to accurately predict the progression of cancer, such as prostate cancer, as well as a means to monitor the effectiveness of treatment with anti-cancer therapies.

The present technology relies on monitoring the protein PSA, which not only results in a high percentage of false positives, but also cannot be used as a predictor of the future progression of the disease. Prostate-specific antigen (PSA) is a serine protease produced by both benign and malignant prostatic epithelium. PSA is presently the single test with the highest positive predictive value for prostate cancer (Catalona, W. J., Richie, J. P., Ahmann, F. R. et al., (1994), J Urol, 151: 1283). However, PSA elevations are not specific for cancer, and PSA may be elevated by other prostate abnormalities, such as benign prostatic hyperplasia (BPH) and prostatitis. Thus, methods have been evaluated to improve the specificity of PSA as a tumor marker for prostate cancer. One of these methods is adjusting PSA levels for prostate volume or PSA density (PSAD). It was proposed by Benson et al that adjusting for PSA prostate volume could help distinguish between men with PSA elevations caused by BPH and those caused by prostate cancer (Benson, M. C., Whang, I. S., Olsson, C. A. et al., (1992), J Urol, 147: 817; Benson, M. C., Whang, I. S., Pantuck, A. et al., (1992), J Urol, 147: 815). However, it was found by Brawer et al that PSAD did not enhance the ability of PSA level alone to predict the presence of cancer in men with PSA values of 4.0 to 10.0 ng/ml and a normal digital rectal examination (DRE) (Brawer, M. K., Aramburu, E. A. G., Chen, G. L. et al, (1993), J Urol, 150: 369).

Another approach being evaluated to improve the specificity of PSA is the use of the complexed PSA (cPSA) assay. It was demonstrated by Stenman et al. (Stenman, U. H., Leinonen, J., Alfthan, H. et al., (1991), Cancer Res, 51: 222), and Lilja et al. (Lilja, H., Christensson, A., Dahlen, U. et al. (1991) Clin Chem, 37: 1618; Lilja, H., Cockett, A. T. K. and Abrahamsson, P. A. Cancer, (1992) 70: 230) that serum from patients with prostate cancer contains a higher proportion of PSA complexed with α₁-antichymotrypsin than serum from patients with BPH. In a large retrospective multi-institutional study by Brawer et al, it was found that using cPSA as a single measurement results in improved specificity for prostate cancer detection compared with total PSA (Brawer, M. K., Cheli, C. D., Neaman, I. E. et al., (2000), J Urol, 163: 1476).

PSA expression is strongly influenced by androgens. Androgens have been found to up-regulate the production of PSA (Young, C. Y. F., Montgomery, B. T., Andrews, P. E. et al., (1991), Cancer Res, 51: 3748; Henttu, P., Liao, S. and Vihko, P., (1992), Endocrinology, 130: 766). Thus, if variations in serum testosterone levels were found to affect serum PSA levels, then testosterone could be used to improve the sensitivity and specificity of PSA. It was previously found by Monath and associates that serum testosterone within the normal range does not correlate with serum PSA (Monath, J. R., McCullough, D. L., Hart, L. J. et al., (1995), Urology, 46: 58).

The increased practice of prostate cancer screening has undoubtedly uncovered many hypogonadal men with elevated PSA. With heightened awareness of the clinical syndrome of hypogonadism, more men are seeking and receiving treatment with testosterone replacement. Testosterone replacement in hypogonadism with elevated PSA is problematic because of the theoretical risk of the exacerbation of occult prostate cancer.

If prostate growth and prostate cancer are simply mediated by androgen levels, one would intuitively expect hypogonadal men to have lower PSA, smaller prostates, and lower risk of prostate cancer. Increased incidence of prostate cancer with age (Hankey, B. F., Feuer, E. J., Clegg, L. X. et al., (1999), J Natl Cancer Inst, 91: 1017), while testosterone levels are decreasing (Feldman, H. A., Longcope, C., Derby, C. A. et al., (2002), J Clin Endocrinol Metab, 87: 589; Harman, S. M., Metter, E. J., Tobin, J. D. et al., (2001) J Clin Endocrinol Metab, 86: 724; Morley, J. E., Kaiser, F. E., Perry, H. M. 3^{rd} et al., (1997), Metabolism, 46: 410,) argues against a “simple” causal relationship. A better understanding of the influence of testosterone on PSA parameters, prostate volume, and likelihood of prostate cancer is needed.

No prior studies have adequately examined the relationship of serum testosterone to PSA in hypogonadal patients presenting for prostate biopsy. In addition, no prior studies have examined the relationship of serum testosterone to cPSA. The present invention is directed to studies that address the relationship between serum testosterone and PSA or cPSA and a means to more accurately detect prostate cancer or diagnose a subject suspected of having prostate cancer.

The citation of any reference herein should not be construed as an admission that such reference is available as “Prior Art” to the instant application.

### SUMMARY OF THE INVENTION

In its broadest aspect, the present invention provides methods of screening for, detecting or diagnosing prostate cancer in a subject comprising determining the level of prostate specific antigen (PSA) and testosterone level in a biological sample from the subject and determining the relationship between the PSA level and testosterone level in order to obtain a testosterone corrected PSA level, or determining the level of prostate specific antigen (PSA) and testosterone in a serum sample, measuring the prostate volume and correcting the PSA level for serum testosterone in order to generate a corrected PSA density (PSAD). A subject having prostate cancer will exhibit a testosterone corrected PSA level (Tc-PSA) and/or a testosterone corrected PSA density (Tc-PSAD) level, which is significantly different from a predetermined range of normal values established from screening normal individuals known to be free of prostate cancer. In a more particular aspect, the methods described herein provide for a more specific and accurate means of detecting prostate cancer in hypogonadal or eugonadal men. The invention also provides methods of identifying individuals at risk for developing prostate cancer, or for determining the effectiveness of anti-cancer therapy. Accordingly, one aspect of the invention provides for a method of detecting prostate cancer in a subject, comprising the steps of:
a) collecting a sample of bodily fluid from a subject suspected of having prostate cancer;
b) determining the level of prostate specific antigen (PSA) and testosterone in the sample; and either:
   i) determining the relationship between the PSA and the testosterone levels in the sample to obtain a testosterone corrected PSA; or
   ii) measuring the prostate volume and relating the PSA level to the prostate volume and serum testosterone to obtain a testosterone corrected PSA density; and
c) comparing the testosterone corrected PSA or the testosterone corrected PSA density (PSAD) to a predetermined range of normal values;

wherein a subject having prostate cancer exhibits a testosterone corrected PSA level or a testosterone corrected PSA density outside the range of normal values

A second aspect of the invention provides a method of screening for, detecting or diagnosing prostate cancer in a subject. In one embodiment, the method comprises the steps of:
a) collecting a sample of bodily fluid from a subject suspected of having prostate cancer;
b) determining the level of testosterone and PSA present in said sample;
c) calculating the prostate volume by ultrasound measurement; and
d) calculating the testosterone corrected PSA (Tc-PSA) value and/or testosterone corrected PSA density (Tc-PSAD) and/or testosterone corrected PSA density of the transition zone (Tc-PSAD-TZ),
   wherein a subject having prostate cancer has a Tc-PSA value and/or a Tc-PSAD and/or a Tc-PSAD-TZ which is significantly different from that obtained from a predetermined range of normal values established from screening normal non-cancerous individuals known to be free of prostatic cancer. In one embodiment, when the level of Tc-PSA and/or Tc-PSAD is determined to be significantly elevated in said bodily fluid compared to a predetermined range of normal values established from screening individuals known to be free of a prostate cancerous condition, the animal subject is identified as being likely to have a hyperproliferative condition or a cancerous condition, in particular, prostate cancer.

In one embodiment, the bodily fluid may be a sample of whole blood, blood cells, serum, plasma, urine or saliva. In another particular embodiment, the testosterone (free or bioavailable testosterone, total testosterone and sex hormone binding globulin), and PSA (free PSA, complexed PSA (cPSA), PRO-PSA, B-PSA, HK2 and total PSA) may be tested using the same sample, or may be tested using different samples collected at the same or different times.

In another particular embodiment, the subject is a human subject and the test determination is performed to segregate subjects that have hyperplasia from subjects that have hyperplasia and cancer. In yet another embodiment, the PSA isoform is selected from the group consisting of free PSA, complexed PSA (cPSA), PRO-PSA, B-PSA, HK2 and total PSA. In yet another embodiment, the testosterone is selected from the group consisting of free or bioavailable testosterone, total testosterone and any testosterone bound-protein such as sex hormone binding globulin (SHBG) for the purpose of testosterone correction of PSA.

In yet another particular embodiment, the testosterone and PSA levels are determined using an immunoassay procedure, such as enzyme linked immunoassay (ELISA) or radioimmunoassay (RIA), although other biochemical or molecular biological means of testing may be used, such as Western or Northern blots, or PCR analysis. In yet another embodiment, the level of testosterone and PSA is determined using a competitive or non-competitive ELISA assay or a sandwich ELISA assay.

A third aspect of the invention provides a means of calculating the testosterone corrected PSA (Tc-PSA) or testosterone corrected PSA density (Tc-PSAD) or testosterone corrected cPSA (Tc-cPSA) or testosterone corrected cPSA density (Tc-cPSAD). In one particular embodiment, the determination of TC-PSA density or Tc-cPSA density is accomplished by dividing the serum PSA or cPSA value by the product of the prostate volume times the serum testosterone value. In another embodiment, any PSA isoform may be measured and corrected for using the testosterone measurement determined as described herein and the prostate volume determinations described herein. The PSA isoform may be selected from the group consisting of free PSA, complexed PSA (cPSA), PRO-PSA, B-PSA, HK2 and total PSA. In yet another embodiment, the testosterone may be selected from the group consisting of free or bioavailable testosterone, total testosterone and any testosterone-bound protein such as sex hormone binding globulin (SHBG) for the purpose of testosterone correction of PSA.

In another particular embodiment, the prostate volume is based on ultrasound measurements, including, but not limited to, transrectal ultrasound measurements (TRUS) taken in the greatest dimension. In yet another particular embodiment, the TRUS measurements are calculated by the ellipsoid volume method of H×W×L×0.52. The volume may be calculated by taking measurements of the entire prostate gland, or alternatively, the volume of the transition zone (TZ)/periurethral benign prostate glandular lobe (adenoma) may also be obtained and the values used to determine PSA density.

A fourth aspect of the invention provides a method of identifying a subject at risk for developing prostate cancer. In one embodiment, the method comprises the steps of:
a) collecting serum from a subject suspected of being at risk for developing prostate cancer;
b) determining the level of testosterone and PSA present in said serum sample;
c) determining the prostate volume by ultrasound measurement; and
d) calculating the testosterone corrected PSA (Tc-PSA) value and/or testosterone corrected PSA density (Tc-PSAD) value and/or testosterone corrected PSA density of the transition zone (Tc-PSAD-TZ),
   wherein a subject suspected of being at risk for developing prostate cancer has a Tc-PSA value and/or a PSA density value which is significantly different from that obtained from a predetermined range of normal values established from screening normal non-cancerous individuals known to be free of prostate cancer.

In one embodiment, the bodily fluid samples are collected at least twice yearly from the subject and it is determined whether the TcPSA or Tc-PSAD measurements change over time compared to baseline values. The measurements obtained are compared to a range of values obtained from subjects free of prostate cancer. Alternatively, the values obtained may be compared to a series of standards established from individuals having prostate cancer and from individuals known to be free of prostate cancer.

In one particular embodiment, the testosterone, PSA and cPSA may be tested using the same bodily fluid sample, or may be tested using different bodily fluid samples. In another particular embodiment, the PSA isoform is selected from the group consisting of free PSA, complexed PSA (cPSA), PRO-PSA, B-PSA, HK2 and total PSA. In yet another embodiment, the testosterone is selected from the group consisting of free or bioavailable testosterone, total testosterone and any testosterone-bound protein such as sex hormone binding globulin (SHBG) for the purpose of testosterone correction of PSA.

In another particular embodiment, the subject is a human subject and the test determination is performed to segregate subjects that have hyperplasia from subjects that have hyperplasia and cancer. In yet another embodiment, the PSA isoform is selected from the group consisting of free PSA, complexed PSA (cPSA), PRO-PSA, B-PSA, HK2 and total PSA. In yet another embodiment, the testosterone is selected from the group consisting of free or bioavailable testosterone, total testosterone and any testosterone-bound protein such as sex hormone binding globulin (SHBG) for the purpose of testosterone correction of PSA.

In yet another particular embodiment, the testosterone and PSA levels as described above are determined using an immunoassay procedure, although other biochemical or molecular biological means of testing known to those skilled in the art may be used.

In yet another particular embodiment the means of calculating the testosterone corrected PSA (Tc-PSA) value or testosterone corrected cPSA density (Tc-cPSAD) value is accomplished by dividing the serum PSA or cPSA value by the product of the prostate volume times the serum testosterone value.

In yet another particular embodiment, the prostate volume (whole gland or transition zone (TZ) ) is based on ultrasound measurements, including, but not limited to, transrectal ultrasound measurements (TRUS) taken in the greatest dimension. In yet another particular embodiment, the TRUS measurements are calculated by the ellipsoid volume method of H×W×L×0.52. In yet another particular embodiment, the prostate volume may be estimated in a non-invasive manner utilizing a combination of PSA, cPSA, free PSA, B-PSA, PRO-PSA and/or human kallekrein (HK2) measurements.

A fifth aspect of the invention provides a method for pre-treatment staging of prostate cancer in a subject having prostate cancer. In one embodiment, the method comprises the steps of:
a) collecting a serum sample from a subject having prostate cancer;
b) determining the level of testosterone and PSA present in said serum sample;
c) determining prostate volume by ultrasound measurement; and
d) calculating the testosterone corrected PSA (Tc-PSA) value and/or testosterone corrected PSA density (Tc-PSAD) value and/or testosterone corrected PSA density of the transition zone (Tc-PSAD-TZ),
   wherein the testosterone corrected PSA (Tc-PSA) value and/or testosterone corrected PSA (Tc-PSAD) density value and/or testosterone corrected PSA density of the transition zone (Tc-PSAD-TZ) is calculated by dividing the PSA value by the product of the prostate volume times the testosterone value.

In one particular embodiment, the testosterone (including free or bioavailable, total or any testosterone-bound protein such as sex hormone binding globulin (SHBG) for the purpose of testosterone correction of PSA), and PSA (including cPSA, free PSA, B-PSA, PRO-PSA and/or human kallekrein (HK2)) may be tested using the same sample of bodily fluid, or may be tested using different samples of bodily fluid.

In another particular embodiment, the subject is a human subject and the test determination is performed to segregate subjects that have hyperplasia from subjects that have hyperplasia and cancer. In yet another embodiment, the PSA is selected from the group consisting of free PSA, complexed PSA (cPSA) and total PSA. In yet another embodiment, the testosterone is selected from the group consisting of free testosterone and total testosterone.

In yet another particular embodiment, the testosterone (including free or bioavailable, total or any testosterone-bound protein such as sex hormone binding globulin (SHBG) for the purpose of testosterone correction of PSA) and PSA levels (including cPSA, free PSA, B-PSA, PRO-PSA and/or human kallekrein (HK2)) are determined using an immunoassay procedure, although other biochemical or molecular biological means of testing known to those skilled in the art may be used.

In yet another particular embodiment the means of calculating the testosterone corrected PSA (Tc-PSA) or testosterone corrected cPSA density (Tc-cPSAD) is accomplished by dividing the serum PSA or cPSA value by the product of the prostate volume times the serum testosterone value.

In yet another particular embodiment, the prostate volume is based on transrectal ultrasound measurements (TRUS) taken in the greatest dimension. In yet another particular embodiment, the TRUS measurements are calculated by the ellipsoid volume method of H×W×L×0.52.

A sixth aspect of the invention provides a kit for measuring one or more isoforms of PSA and one or more forms of testosterone, wherein the PSA isoform is selected from the group consisting of free PSA, cPSA, Pro-PSA, B-PSA, HK2 and/or total PSA and the forms of testosterone are selected from the group consisting of free/bioavailable testosterone, total testosterone and any testosterone-bound protein such as sex hormone binding globulin (SHBG) for the purpose of testosterone correction of PSA, in a subject comprising:
a) a solid substrate comprising an immobilized binding partner specific for at least one or more PSA isoform and at least one or more form of testosterone; and
b) either:
   i) an enzyme conjugated second binding partner capable of binding to the PSA isoform of step a) and testosterone; or
   ii) a biotinylated second binding partner capable of binding to the PSA isoform of step a) and testosterone; and
c) either:
   i) an enzyme substrate and a developing reagent specific for the enzyme conjugated second binding partner of step b) i); or
   ii) a streptavidin conjugated third binding partner specific for the second binding partner of step b) ii); and
d) a buffer for washing and sample dilution; and
e) a standard for a PSA isoform and testosterone; and
f) instructions for using the kit.

In one embodiment, the kit may contain all of the reagents necessary to measure PSA and testosterone, at the same time. In one embodiment, the kit may contain reagents necessary to measure PSA selected from the group consisting of free PSA, complexed PSA (cPSA), PRO-PSA, B-PSA, HK2 and total PSA. The kit may contain the reagents necessary to measure testosterone selected from the group consisting of free or bioavailable testosterone, total testosterone and any testosterone-bound protein such as sex hormone binding globulin (SHBG) for the purpose of testosterone correction of PSA. The assay kit may be formatted for use as a competitive or non-competitive ELISA assay. Alternatively, the kit may be structured in much the same way as a take home pregnancy kit, for example, using a test strip format. The kit may also contain binding partners, for example, primary antibodies specific for PSA selected from the group consisting of free PSA, complexed PSA (cPSA), PRO-PSA, B-PSA, HK2 and total PSA and testosterone selected from the group consisting of free or bioavailable testosterone, total testosterone and any testosterone-bound protein such as sex hormone binding globulin (SHBG) for the purpose of testosterone correction of PSA, and secondary antibodies that are conjugated to an enzyme or radioactive marker for easy detection of binding. The kit may also contain the reagents to detect the binding of the primary or secondary antibodies, as well as standards with which to compare the final readout to determine whether the test sample falls within or outside of the normal range.

Other advantages of the present invention will become apparent from the ensuing detailed description.

### DETAILED DESCRIPTION

Before the present methods and treatment methodology are described, it is to be understood that this invention is not limited to particular methods, and experimental conditions described, as such methods and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the particular methods and materials are now described. All publications mentioned herein are incorporated herein by reference in their entirety.

Definitions

The terms used herein have the meanings recognized and known to those of skill in the art; however, for convenience and completeness, particular terms and their meanings are set forth below.

By “patient” or “subject” is meant a human or non-human mammal that may benefit from the therapies described in the present application, for example, anti-cancer therapies.

By “effectiveness of therapy” is meant that upon treating a subject having prostate cancer with anti-cancer therapy, one can determine whether the treatment has resulted in the desired outcome. For example, in the case of treating a patient having prostate cancer with chemotherapy or radiation therapy or with any standard form of therapy approved for treatment of such conditions, one may observe a decrease not only in the amount of PSA, but also a decrease in the progression of tumor growth, and perhaps in certain cases, a remission in the cancer and prevention of metastasis.

“Surrogate biomarker” or “biomarker” or “marker” as used herein, refers to a highly specific molecule, the existence and levels of which are causally connected to a complex biological process, and reliably captures the state of said process. Furthermore, a surrogate biomarker, to be of practical importance, must be present in samples that can be obtained from individuals without endangering their physical integrity or well-being, preferentially from biological fluids such as blood, plasma, urine, saliva or tears. The biomarkers of prostate hyperplasia or prostate cancer, as used herein, include increased PSA, cPSA, pro-PSA, B-PSA, HK2, Tc-PSA and Tc-cPSA. The levels of these biomarkers should reflect the degree of tumor cell proliferation or tumor burden in the body. Furthermore, the presence of these biomarkers, in particular, Tc-PSA, Tc-cPSA, pro-PSA, B-PSA and HK2 should reflect the need for anti-cancer therapy. The normalization of these biomarkers may also reflect the effectiveness of anti-cancer therapy as provided in the present application. These markers may also help in pre-treatment staging. When used alone, or more preferably in conjunction with the methods described herein, they may aid in determining the aggressiveness of the tumor or tumor recurrence.

A person “suspected of being in need of such treatment” may refer to an individual suffering from symptoms suggestive of tumor growth.

A person “at risk for developing prostate cancer” in terms of the methods of the present invention may refer to an individual suffering from symptoms suggestive of tumor growth or due to the age of the individual or family history of the individual may be prone to developing such condition. Methods of assessing the risk of an individual for developing prostate cancer include obtaining information related to the family history of the individual, ethnic background, age and serum PSA levels.

“Staging” refers to assessment of the volume of the disease, either pre-treatment or post-treatment, or a point within the natural history of the disease at which the patient presents to the physician. Staging establishes how far along the cancer has progressed in its natural history and may be assessed by tissue biopsy. Following an assessment of the biopsied tissue, staging is done through use of published nomograms in conjunction with determination of the Gleason score.

“Diagnosis”, “diagnosing”, “screening” or “detecting” refers to diagnosis, prognosis, monitoring, characterizing, selecting patients, including participants in clinical trials, and identifying patients at risk for or having a particular disorder or clinical event or those most likely to respond to a particular therapeutic treatment, or for assessing or monitoring a patient's response to a particular therapeutic treatment.

“PSA” as used herein, refers to “prostate specific antigen”, an enzyme normally secreted by the prostate, which is a glycoprotein of 28,700 Daltons, consisting of 237 amino acid residues produced primarily by the epithelial cells lining the acini and ducts of the prostate gland (McCormack R T et al., Urolozy 45,729-744, 1995). (Acini are parts of any gland where fluid is produced.) PSA may exist as “free PSA”, which means it is not bound/complexed to any protein, or it may exist as “complexed PSA” which indicates it is bound to a protein such as that described below. PSA is concentrated in prostatic tissue, and serum PSA levels are normally very low. Disruption of the normal prostatic architecture, such as by prostatic disease, allows greater amounts of PSA to enter the general circulation.” From Prostate Specific Antigen (PSA) Best Practice Policy, Oncology Vol 14, No 2 (February 2000) ( American Urological Association). High PSA levels in the blood may be a sign of any prostate problem. PSA rise may indicate infection. It may indicate benign growth or swelling of the prostate. Or it may indicate prostate cancer. PSA exists in three major forms in the blood: 1) free (PSA-f), 2) bound to a protein called alpha-1-antichymotrypsin (PSA-ACT), and 3) bound to another protein called alpha-2 macroglobulin (PSA-MG). People with prostate cancer have more of the form bound to alpha-1-antichymotrypsin and less of the free form than do healthy men or those with benign diseases of the prostate.

“cPSA” refers to “complexed PSA” which is PSA that is complexed or bound to any other protein including, but not limited to, alpha-1-antichymotrypsin (ACT), alpha-1-protease inhibitor (API), or alpha-2-macroglobulin in a patient's blood sample. PSA bound to ACT accounts for about 60-95% of the total PSA, whereas PSA bound to alpha-1-protease inhibitor accounts for about 1-2% of total PSA. Recent data suggests that this test is more sensitive and specific than the PSA test, thus improving the overall accuracy of the test for prostate cancer.

“Total PSA” refers to the measurement of both free PSA and complexed PSA, as defined herein.

“Free testosterone” refers to the testosterone which circulates in the bloodstream in a form which is unattached to sex hormone binding globulin (SHBG). A major fraction of testosterone is specifically bound with high affinity and low capacity to sex-hormone-binding globulin (SHBG), whereas most of the remaining testosterone is bound with low affinity to albumin (ALB), leaving only 1-2% to circulate as “free” testosterone (FT) not bound to protein in serum (also known as bioavailable testosterone).

“Total testosterone” refers to the amount of testosterone present in a subject taking into account the combination of both free testosterone and testosterone attached to its carrier molecules, sex hormone binding globulin (SHBG) or albumin (ALB). When the PSA level is corrected for testosterone, the correction may be made using total testosterone, or free or bioavailable testosterone or SHBG.

“Transrectal ultrasound” or “TRUS” is a 5- to 15-minute outpatient procedure that uses sound waves to create a video image of the prostate gland. The procedure involves the placement of a small, lubricated probe into the rectum which releases sound waves, which create echoes as they enter the prostate. Prostate tumors often create echoes that are different from normal prostate tissue. The echoes that bounce back are sent to a computer that translates the pattern of echoes into a picture of the prostate. TRUS is used to estimate the weight of the prostate gland, helping doctors get a better idea of PSA density, which helps distinguish benign prostatic hyperplasia (BPH) from prostate cancer.

“Ellipsoid volume method for measuring prostate volume” is a method for assessing the volume of the prostate and is an important and integral part of the TRUS procedure. Several formulas have been used, but the most common one is the ellipsoid formula, which requires measurement of 3 prostate dimensions. Dimensions are first determined in the axial plane by measuring the transverse and anteroposterior dimension at the estimated point of widest transverse dimension. The longitudinal dimension is measured in the sagittal plane just off the midline because the bladder neck often obscures the cephalad extent of the gland. The ellipsoid volume formula is then applied, as follows:
Volume=height×width×length×0.52

The prostate volume may be calculated by measuring the whole gland and insertion of the measurments of height, width and length into the formula above, and the value obtained is used to aid in determination of PSA density. Alternatively, the volume of the transition zone/periurethral benign prostate glandular lobe (adenoma) may also be obtained and the values used to determine PSA density.

“PSA density” or “PSAD” refers to the PSA value obtained by dividing the PSA value from the sample of bodily fluid by the prostate volume (determined by the methods described herein, such as for example, by using the ellipsoid volume formula). For example:

$$PSA density ( PSD ) = \frac{PSA value}{prostate volume}$$

The “PSA density of the transition zone” or “PSAD-TZ” refers to the PSA density obtained by dividing the PSA value from the sample of bodily fluid from the patient by the volume of the transition zone, which is obtained by ultrasound measurements, such as those described herein. For example:

$$\begin{matrix}PSA density of the \\ transition zone ( PSAD \text{-} TZ )\end{matrix} = \frac{PSA value}{transition zone volume}$$

“Testosterone corrected PSA density” or “Tc-PSAD” refers to the PSA value obtained by dividing the PSA value from the patient's sample of bodily fluid by the product of the prostate volume (determined by the methods described herein, for example, by using the ellipsoid volume formula) times the testosterone value obtained from a biological sample from the patient. That is:

$$\begin{matrix}Testosterone Corrected \\ PSA density\end{matrix} = \frac{PSA value}{( prostate volume \times Testosterone )}$$

“Testosterone corrected PSA density of the transition zone” or “Tc-PSAD-TZ” refers to the PSA density obtained by dividing the PSA value from the patient's sample of bodily fluid by the product of the transition zone volume (determined by the methods described herein), times the testosterone value obtained from a biological sample from the patient. That is:

$$\begin{matrix}\begin{matrix}\begin{matrix}Testosterone Corrected \\ PSA density\end{matrix} \\ of the transition zone\end{matrix} \\ ( Tc \text{-} PSAD \text{-} TZ )\end{matrix} = \frac{PSA}{( \begin{matrix}transition zone volume \times \\ Testosterone\end{matrix} )}$$

Testosterone corrected PSA may be calculated using either PSA, cPSA, pro-PSA, B-PSA, HK2 and total or free/bioavailable testosterone or any testosterone-bound protein such as sex hormone binding globulin (SHBG) for the purpose of testosterone correction of PSA.

“Testosterone correction” of PSA or PSA density refers to any formula utilizing free testosterone, total testosterone or bioavailable testosterone or any testosterone-bound protein such as sex hormone binding globulin (SHBG) for the purpose of testosterone correction of PSA to improve the specificity and/or accuracy of PSA, cPSA, free PSA, B-PSA, Pro-PSA or their respective density derivations in detection or staging of prostate cancer. Thus, “testosterone corrected PSA” refers to the PSA level that is obtained by taking into account the relationship between the PSA value and the testosterone value obtained in a sample of bodily fluid.

“Sensitivity” is defined as the capability of a test to identify the presence of disease expressed as the ratio of true positives to the sum of true positives and false negatives.

“Specificity” is defined as the capability of a test to identify the absence of disease as the ratio of true negatives to the sum of true negatives and false positives.

“B-PSA” or “benign PSA” refers to an isoform of free PSA, which is associated with benign prostatic hyperplasia (BPH) and is found predominantly in the transition zone of the prostate in patients with BPH.

“Pro-PSA” refers to a precursor isoform of free PSA, which is associated with prostate cancer. ProPSA is comprised of native proPSA as well as truncated proPSA forms, [-2]pPSA and [-4]pPSA, which have been shown to be more cancer associated than native proPSA. It may be useful in identifying the more aggressive forms of prostate cancer.

“HK2” or “kallekrein 2” refers to an enzyme in the PSA family that may convert free PSA to bound PSA. HK2 levels, while they are in the blood at very low concentrations, do increase with the presence of cancer. Since the percentage of HK2 is higher and free PSA is lower when cancer is present, a ratio of the two may help in distinguishing between BPH and cancer. Measurement of HK2 may also help in predicting tumor stage.

“Pre-treatment staging” refers to the pathological stage to which the tumor has developed prior to the initiation of therapy. Means for staging a prostate tumor are known to those skilled in the art. The likelihood of effective prostate cancer treatment by radical prostatectomy or radiation therapy is dependent on the pathologic stage to which the tumor has developed at the time of treatment. Staging is used to aid physicians in making treatment decisions. The methods described in the present application may be used as a reflex test to aid in development of treatment strategies.

General Description

The present invention provides methods of screening for, detection, diagnosis, or prognosis of prostate cancer. The method further provides for improvements in the current means for detecting prostate cancer by combining the standard tests for measuring prostate specific antigen (PSA) with a correction for serum testosterone levels. Moreover, the methods of the present invention include screening for levels of PSA, complexed PSA (cPSA), free PSA, PRO-PSA, B-PSA, HK2 and total PSA and correcting these values with levels of free/bioavailable testosterone, and/or total testosterone and/or sex hormone binding globulin. The correction for serum testosterone may be achieved by dividing the serum PSA value by the product of the prostate volume times the serum testosterone value. Moreover, the determination of PSA and/or testosterone may be achieved by using standard procedures including immunoassay procedures, such as enzyme-linked immunosorbent assays (ELISA) or radioimmunoassays. The present invention also involves the determination of prostate volume by ultrasound methods such as, but not limited to, transrectal ultrasound (TRUS) procedures, whereby the measurements are taken in the greatest dimension. The ellipsoid volume method may be used to make these determinations, using the following formula:
Height×Width×Length×0.52

Moreover, the present invention contemplates that the methods of the invention may be used as a reflex test to be used as a secondary screen for testing patients who exhibit an abnormal primary screen, such as with the PSA test. Accordingly, when a patient presents with an abnormal PSA level, the sample of the patient's bodily fluid will be used for measuring testosterone levels and the calculation of the testosterone corrected PSA levels will help provide better specificity to aid in determining whether that patient has prostate cancer rather than benign prostate hyperplasia.

Prostate Specific Antigen (PSA)

The prostate specific antigen (PSA) is a glycoprotein of 28,700 Daltons, consisting of 237 amino acid residues mainly secreted by the prostate gland secretory luminar cells lining the secretory duct. (McCormack R T et al., Urolozy 45,729-744, 1995).

Small quantities of PSA are normally found in the circulatory system. The amount of serum PSA can increase as carcinomas of the prostate develop and mature. Elevated serum PSA levels have been used to aid in the diagnosis and monitoring of prostate cancer, for example, for the early detection of prostate adenocarcinoma. (Rittenhouse, H. G. et al., Critical Reviews in Clinical Laboratory Sciences, 35(4), 275-368, 1998).

As is conventionally understood or practiced in the field of urology, men having serum PSA concentrations less than 2 ng/ml generally are not diagnosed with, or considered to have, prostate cancer. However, when serum PSA concentration levels increase, the likelihood of being diagnosed with prostate cancer increases. For example, typically, 22-27% of men that have a PSA level of 2.5 -4.0 ng/ml are diagnosed with prostate cancer (Catalona et al. (1997), J. American Medical Association, 277(18): 1452-1455; Okihara et al. (2001), J. Urology 165(6): 1930-36). When the concentration of serum PSA is between 4 and 10 ng/ml, one in four men will be diagnosed with prostate cancer, and when the levels increase above 10 ng/ml, the ratio is one out of two men. (Catalona, W. J. et al., Journal of the American Medical Association, 274(15), 1214-1220, 1995 Catalona, W. J. et al., J. Urol., 151, 1283-1290, 1994; Brawer, M. K. et al., J. Urol., 147, 841-845, 1992; Oesterling, J. F., J. Urol., 145, 907-923, 1991).

Existing immunoassay systems used to detect or monitor prostate cancer incorporate one or more monoclonal antibodies (mAbs) capable of binding to any of the six different known major PSA epitopes (see Stenman U. H. et al. Tumor Biology, 20, suppl. 1, 1-12, 1999).

Generally, immunoassays can be categorized into quantitative or qualitative groups, as discussed below. The quantitative type of immunoassay is typically more expensive and relatively difficult to conduct. The qualitative immunoassays are relatively less expensive and easier to perform, but do not necessarily provide the amount or accuracy of information obtained with the quantitative immunoassays.

With respect to the quantitative type of immunoassays, conventionally known as “sandwich assays” and as conventionally practiced, one antibody is coupled to a solid support, and a second antibody is coupled to a detectable label. A test antigen having separate binding sites (epitopes) for the first and second antibodies is exposed to the antibody coupled to the solid support such that the antigen binds to that antibody. Subsequently, the labeled second antibody is added to the support to permit the binding of the labeled second antibody to the test antigen. Thus, the amount of the antigen present in a sample is a function of the amount of detected label bound to the second antibody bound to the antigen. Examples of such detectable labels include chromophores, radioisotopes, or enzymes which can be converted into a product that can be measured photometrically. When the amount of detected label is compared to the amount of antibody binding in a standard sample containing known amounts of antigen, quantitative results can be obtained. However, as indicated above, this procedure is typically complicated, time consuming, and expensive to perform compared to immuno-chromotography techniques described below, because this assay requires personnel training, complicated instruments, and test samples or standards, to be used during each measurement or assay.

To attempt to reduce the difficulty and expense of the quantitative immunoassay described above, immuno-chromatography methods have been developed. These tests provide qualitative information (e.g. a positive or negative result). The immuno-chromatography method typically utilizes a solid support such as a membrane strip having a region (a “reaction zone”) coated with a first antibody (a “capture antibody”) that is capable of binding to an antigen. The concentration of the capture antibody is empirically determined prior to the manufacture of the device. The concentration of the capture antibody is typically selected based on antibody/antigen binding data corresponding to the detection of an antigen above a single selected concentration threshold. The concentration threshold (i.e., the concentration of antigen that is believed to correlate with a disease condition) is chosen based on clinical or research data used in the diagnosis and/or monitoring of diseases having disease specific antigens. As discussed herein, clinicians typically begin to carefully monitor male patients for prostate cancer when their serum PSA levels are greater than 2.5 ng/ml. Thus, existing immunochromatography assays utilize a concentration of the capture antibody in the reaction zone that permits detection of PSA above a 2 ng/ml concentration threshold. A second antibody (a “detection antibody”), capable of binding the antigen at a different site, or epitope, from the first antibody, is usually coupled with color particles, such as colloidal gold or blue latex, and is applied in a solution having other factors, such as detergents, to facilitate solubilization of the labeled antibody onto a different region (a “reagent zone”) of the solid support, e.g., near one end of the membrane strip. The sample is then loaded on the membrane near the end that contains the detection antibody. The sample subsequently diffuses through the region with the detection antibody where the antigen binds to the detection antibody, and diffuses continuously toward the region of the capture antibody. Because the detection antibody is applied to the reagent zone with solution components that increase solubilization of the antibody, the detection antibody is capable of diffusing with the antigen as it diffuses towards the other end of the membrane strip. When the antigen bound by the detection antibody interacts with the capture antibody, it is trapped in the reaction zone. If the test antigen present in the solution does not recognize the capture antibody, or it is present at a concentration lower than the concentration threshold determined by the capture antibody, the test antigen coupled with the detection antibody with the color particles will not bind to the membrane strip region containing the capture antibody, and thus, no staining will be present in the reaction zone, indicating that the antigen is present at a concentration less than the concentration threshold of the capture antibody (i.e. 2 ng/ml). Similarly, if no antigen is present in the sample, no binding will occur with either of the antibodies, and thus, no staining will occur. Therefore, a positive result is indicated by the presence of color in the reaction zone. The intensity of color correlates with the amount of bound antigen in the reaction zone. Thus, it is possible that the user will be able to make a more quantitative interpretation based on the degree of staining intensity.

However, as indicated above, these known immuno-chromatography-based PSA antibody assays only provide information of PSA concentration above a single value, or concentration threshold, for example 2 ng/ml, based on the clinical values discussed above, and do not provide multiple values or concentration thresholds, in a single test, to facilitate more accurate measurement of PSA concentration in a single test.

Due to the limitation of using PSA measurement for discriminating prostate cancer from other benign prostatic diseases in which serum PSA values are slightly elevated, the detection of PSA forms such as free PSA and PSA-[alpha]1 -antichymotrypsin complex (PSA-ACT) has been reported to improve the specificity (Kuriyama, M., (1994), Int. J. Urol., 145:99-113; Kuriyama, M. et al. (1999), Japanese J Clin. Oncol. pp.303-307)). Moreover, the ratio of free to total PSA (F/T) has also been considered to be more useful for the detection of prostate cancer in the gray zone PSA group (Catalona, W J et al. (1995), J. Am. Med. Assoc. 274:1214-1220). However, the cut-off values for F/T vary (Catalona, W J et al. (1995), J. Am. Med. Assoc. 274:1214-1220; Luderer, A A et al., (1995), Urology 46:187-194; Froschermaier, SE et al., (1996), Urology 47:525-528).

The more recently developed commercial assays use different techniques to measure PSA. Some are immunoradiometric, some are enzyme immunoassays and one is a chemiluminescent immunoassay. For example, several standardized radioimmunoassay (RIA) and enzyme linked immunosorbent (ELISA) assay kits are available for measurement of PSA. These include kits manufactured by Yang Laboratories (Bellevue, Wash.), Hybritech (San Diego, Calif.), DPC (Choba, Japan) (Kuriyama, M. et al. (1998), Jpn J Clin Oncol 28:661-665), Abbott (Chicago, Ill.), Roche (Basel, Switzerland), and Bayer (New York, N.Y.) (Allard, W J, (1998), Clin Chem 44:1216-1223; Brawer, M K, (1998), Urology 52:372-378). Hybritech's monoclonal antibody to PSA has been adapted to the Stratus™ analyzer (Baxter-Dade, Miami, Fla.) in a fluorescence immunoassay (FIA) system and has received FDA approval. PSA has recently been adapted to the IMx™ analyzer (Abbott, Abbott Park, Ill.) and this assay is also FDA approved (Smith, A. et al. (1990), Clin. Chem. 36:1096; Sampson, M. et al. (1992), Clin. Chem, 38:949; Vessella, R. et al. (1991), Clin. Chem. 37: 1024; Goldberg, J M. et al. (1992), Clin. Chem. 38: 975; Vessella, R L, (1992), Clin. Chem. 38:2044-54). Alternatively, PSA may be measured using a quantitative reverse transcription polymerase chain reaction (RT-PCR) assay as described by Ylikoski et al., which provides sensitive and quantitative detection of PSA mRNA expression from blood samples (Ylikoski, A. et al. (Clin. Chem. (1999), 45(9): 1397-1407). The Hybritech Inc Tandem-R assay, which was approved by the FDA for detection of PCA, is a solid-phase, two site, monoclonal antibody immunoradiometric assay. In this assay method, the PSA in serum binds to a unique monoclonal antibody fixed on a plastic bead. Simultaneously, a separate distinct epitope of the PSA molecule is detected with a second radiolabeled monoclonal antibody. Six calibrators are used in this test at different concentrations covering the range of the test. Radioactivity is quantitated using a gamma ray counter and concentration is calculated from a standard reference curve using a plot of total counts per minute versus the log of the dose (ng/ml), connecting a straight line between each of the calibrator points.

As a general rule, perhaps the real value of the PSA test for early detection of prostate cancer can be appreciated by taking into account the change in value over time. Thus, by measuring the PSA levels for example, on a yearly basis, any incremental change of 0.75 ng/ml in a year should be investigated further (Carter et al. JAMA, (1992), Vol. 267:2236-2238).

Furthermore, the PSA should be monitored after radical prostatectomy, since the presence of higher than normal levels of PSA is evidence of residual disease, tumor metastasis, or of disease recurrence.

Testosterone: General Aspects and Methods of Measurement

Testosterone, which is the principal androgen in men, is found in the circulation and is distributed in free, weakly-bound, and tightly-bound forms. A number of analytical methods have been developed for measuring these various forms of testosterone (Pearce, S. et al. (1989), Clin. Chem. 35/4:632-635; Klee, G G, (2000), Mayo Clin. Proc. 75(Suppl):S19-S25; Wheeler, M J, (1995), Ann. Clin. Biochem. 32(4):345-357; Vermeulen, A. (1999), J. Clin. Endocrinol. Methods, 84(10):3666-3672; Wilke, T J, (1987), Clin. Chem., 33(8):1372-1375; Barini, A., (1993), Clin. Chem. 39(6):938-941; Vlahos, I. (1982), Clin. Chem. 28(11):2286-2291; Cheng R W, (1986), Clin. Chem. 32(7):1411; Dechaud, H., (1989), Clin. Chem. 35(8):1609-1614; Ooi, D S, (1999), Clin. Chem. 45(5):715; Dabbs, J M, (1995), 41(11):1581-1584). Free testosterone can be determined by methods such as equilibrium dialysis, equilibrium ultra filtration, and analogue immunoassay methods. The concentration of physiologically active testosterone can be also be estimated by calculation of free androgen index or by the measurement of bioavailable or salivary testosterone.

Testosterone levels change dramatically during the life cycle of males (Ismail, A A A, (1986), Ann. Clin. Biochem. 23:113-134; Gronowski, A M, In Burtis CA, Ashwood, E R, eds. Tietz Textbook of Clinical Chemistry, 3^{rd} ed., Philadelphia, Pa.: WB Saunders: 1999:1601-1641). Testosterone concentrations in the male fetus rise at approximately 12 weeks after conception due to the stimulatory effect of human chorionic gonadotropin (hCG) on the developing testes. While testosterone levels fall to low levels by the third trimester of pregnancy, they start increasing again in the male neonate after about three weeks of life, nearly reaching adult levels by the age of two months. Levels then gradually fall to less than 0.3 ng/mL by six months and remain at low levels until puberty (Wheeler, M J, (1995), Ann. Clin. Biochem. 32(4):345-357). In females, testosterone levels remain low from conception until puberty (Wheeler, M J, (1995), Ann. Clin. Biochem. 32(4):345-357). (Wheeler, M J, (1995), Ann. Clin. Biochem. 32(4):345-357). During puberty, testosterone levels in females increase to adult levels, but they never come close to the levels of adult males. Testosterone levels in males rise during puberty to the lifetime maximum levels achieved in young adulthood. As many men and women age, their testosterone levels gradually decrease to levels that are less than 50% of the maximal levels achieved during young adulthood (Leifke, E. et al. (2000), Clin. Endocrinol. 53: 689-695; Davis, S R, (1997), Curr. Opin. Obstet. Gynecol. 9:177-180). Like other steroid hormones, testosterone and DHT, initiate their physiologic actions by forming complexes with specific cytoplasmic receptors within the cells of target tissues. These complexes then enter the nucleus and cause changes in gene transcription and protein synthesis.

The form of testosterone that is tightly bound to plasma proteins is not able to enter cells and produce androgenic effects. Only about 2% of the total testosterone in the plasma of men, and about 1% of the total testosterone in women is free or nonprotein bound (Wheeler, M J, (1995), Ann. Clin. Biochem. 32(4):345-357). In most men and women, more than 50% of total circulating testosterone is bound to sex hormone-binding globulin (SHBG), and most of the rest is bound to albumin (Wheeler, M J, (1995), Ann. Clin. Biochem. 32(4):345-357). Because of the high affinity of SHBG for testosterone, SHBG-bound testosterone is not readily available for intracellular complex formation (Wheeler, M J, (1995), Ann. Clin. Biochem. 32(4):345-357). For this reason, testosterone-bound SHBG is considered biologically inactive. Although albumin has a much lower binding affinity for testosterone, it binds a significant portion of the total testosterone because it is present at much higher plasma concentrations than SHBG (Wheeler, M J, (1995), Ann. Clin. Biochem. 32(4):345-357; Manni, A. (1985), J. Clin. Endocrinol. Metab. 61(4):705-710). The rapid dissociation of weakly-bound testosterone from albumin, together with a relatively long transit time of albumin through target tissue capillary beds, results in the availability of essentially all albumin-bound testosterone for steroid-receptor interaction (Manni, A. (1985), J. Clin. Endocrinol. Metab. 61(4):705-710). The sum of the free and albumin-bound testosterone is often referred to as bioavailable testosterone (Lobo, R A, (2000), Ann. Intern. Med. 132(12):989-993).

There are many methods for measuring testosterone levels (Klee, G G, (2000), Mayo Clin. Proc. 75(Suppl):S19-S25; Wheeler, M J, (1995), Ann. Clin. Biochem. 32(4):345-357; Vermeulen, A. (1999), J. Clin. Endocrinol. Metab. 84:(10:3666-3672). In addition, there are numerous schools of thought as to which form of the hormone should be measured and which analytical method provides the most accurate assessment of biological activity. While there may not be a clear consensus on this issue, it is important to understand the analytical basis for the various methods available. Some of the different approaches currently used for measuring testosterone status include (1) total testosterone, (2) androgen index calculation, (3) Free testosterone by equilibrium dialysis or equilibrium ultrafiltration, (4) free testosterone by analog tracer immunoassay, (5) bioavailable testosterone, and (6) salivary testosterone. Each is described below.

Total Testosterone

It is common for most clinical laboratories performing total testosterone testing to use automated methods based on immunoassay (Klee, G G, (2000), Mayo Clin. Proc. 75(Suppl):S19-S25; Gronowski, A M, In Burtis C A, Ashwood, E R, eds. Tietz Textbook of Clinical Chemistry, 3^{rd} ed., Philadelphia, Pa.: WB Saunders: 1999:1601-1641). The first step in this procedure is to displace the bound testosterone from SHBG and albumin. There are various means of achieving this, such as the addition of low-pH buffers, surfactants, salicylates, or a competing steroid that does not bind to the anti-testosterone antibody used in the immunoassay. The testosterone antisera used in commercial methods may sometimes cross-react to some extent with other steroids, in particular DHT. To minimize this problem, solvent extraction and/or chromatography have been used to remove these interfering compounds prior to testosterone measurement. Unfortunately, these pre-purification techniques cannot be readily incorporated into methods utilizing automated analyzers. Fortunately, plasma levels of DHT are only about one tenth of testosterone levels, and the cross-reactivity is typically less than 5% (Klee, G G, (2000), Mayo Clin. Proc. 75(Suppl):S19-S25). However, in the majority of cases, the interferences that are observed in these commercial assays do not detract from the clinical utility of the results generated (Klee, G G, (2000), Mayo Clin. Proc. 75(Suppl):S19-S25).

Androgen Index Calculation

The concentration of testosterone in the various free and bound forms is essentially a function of total testosterone concentration and the relative concentrations of SHBG and albumin. Generally, it can be predicted that increased SHBG will decrease the concentration of both free and bioavailable testosterone for a given total testosterone concentration. Thus, many clinicians use a calculated free androgen index to estimate physiologically active testosterone (Wheeler, M J, (1995), Ann. Clin. Biochem. 32(4):345-357; Gronowski, A M, In Burtis C A, Ashwood, E R, eds. Tietz Textbook of Clinical Chemistry, 3^{rd} ed., Philadelphia, Pa.: WB Saunders: 1999:1601-1641). This index is typically calculated as the ratio of total testosterone divided by SHBG and multiplied by 100 to yield numerical results comparable in free testosterone concentration (Wheeler, M J, (1995), Ann. Clin. Biochem. 32(4):345-357; Robinson. S. et al., (1992), Br. J. Obstet. Gynecol. 99(3):232-238; Vermeulen, A. (1999), J. Clin. Endocrinol. metab. 84(10):3666-3672; Blight, L F, (1989), Ann. Clin. Biochem. 26: 311-316). Alternatively, more complicated mathematical algorithms can be used to estimate the percentage of free testosterone from the SHBG concentration alone or in combination with albumin concentration (Wheeler, M J, (1995), Ann. Clin. Biochem. 32(4):345-357; Gronowski, A M, In Burtis C A, Ashwood, E R, eds. Tietz Textbook of Clinical Chemistry, 3^{rd} ed., Philadelphia, Pa.: WB Saunders: 1999:1601-1641). The precision of these algorithms is subject to the combined errors of the individual tests performed but a number of authors have shown them to be useful in the assessment of testosterone status Wheeler, M J, (1995), Ann. Clin. Biochem. 32(4):345-357; Robinson. S. et al., (1992), Br. J. Obstet. Gynecol. 99(3):232-238.

Free Testosterone by Equilibrium Dialysis or Equilibrium Ultrafiltration

Because the concentration of free testosterone is very low in serum (generally less than 2% of the total testosterone concentration), its measurement is technically challenging. Since the assay methods that are generally used are not sensitive enough to quantitate free testosterone directly, free testosterone is often estimated by indirect methods. These methods require the addition of tritiated testosterone to the sample, which is allowed to come to equilibrium with testosterone in the serum at physiological temperature (Wheeler, M J, (1995), Ann. Clin. Biochem. 32(4):345-357; Gronowski, A M, In Burtis C A, Ashwood, E R, eds. Tietz Textbook of Clinical Chemistry, 3^{rd} ed., Philadelphia, Pa.: WB Saunders: 1999:1601-1641). It is imperative that the amount of the added radiolabled testosterone be low enough so that the addition will not significantly increase the total testosterone concentration. Once equilibrium is achieved the free testosterone is separated from the bound testosterone by filtration through a membrane. This filtration can be accomplished by equilibrium dialysis (Robinson. S. et al., (1992), Br. J. Obstet. Gynecol. 99(3):232-238; Wilke, T J, (1987), Clin. Chem. 33(8):1372-1375) or by centrifugal ultrafiltration (Barini, A. (1993), Clin. Chem. 39(6):938-941). The percentage of free testosterone is calculated using the measurement of the radioactivity in the protein-free ultrafiltrate. The concentration of free testosterone is then calculated by multiplying the percentage of free testosterone by the total testosterone concentration. Measurement of free testosterone by these methods is not available in most clinical laboratories due to the complicated nature of the testing and the requirement of a scintillation counter to measure the tritiated testosterone concentration. The results of equilibrium dialysis and centrifugal ultrafiltration methods have been shown to be quite comparable (Barini, A. (1993), Clin. Chem. 39(6):938-941). Equilibrium dialysis is often considered to be the “gold standard,” however, centrifugal ultrafiltration is somewhat simpler to perform and may theoretically be more accurate due to the fact that the equilibrated sample is not diluted with dialysis buffer (Gronowski, A M, In Burtis C A, Ashwood, E R, eds. Tietz Textbook of Clinical Chemistry, 3^{rd} ed., Philadelphia, Pa.: WB Saunders: 1999:1601-1641).

Free Testosterone by Analog Tracer Immunoassay.

There are a number of commercial kits available for the direct estimation of free testosterone in serum. These kits use a labeled testosterone analogue that has a low binding affinity for both SHBG and albumin but is bound by antitestosterone antibody used in the assay. The unbound analogue present in the plasma competes with free testosterone for binding sites on an antitestosterone antibody that is immobilized on the surface of the well or assay tube. The first kits developed used a radiolabled testosterone analogue to compete with free testosterone for binding sites on an antibody-coated polypropylene tube (Vlahos, I., (1982), Clin. Chem. 28(11):2286-2291). More recently developed assay kits utilize an enzyme-labeled analogue that can be measured after competitive binding to antitestosterone antibodies coated on the wells of microtiter plates. These analogue methods are technically less demanding than equilibrium dialysis or centrifugal ultrafiltration. An added feature is that these assays require substantially less sample. The analogue methods also offer the advantage of direct estimation of free testosterone concentration without the need to measure total testosterone. Another added advantage of the enzymatic methods, which allows them to be readily performed by many laboratories, is the fact that they are nonisotopic.

Bioavailable Testosterone

Bioavailable testosterone is a term applied to the sum of circulating free testosterone and albumin-bound (weakly bound) testosterone (Wheeler, M J, (1995), Ann. Clin. Biochem. 32(4):345-357; Gronowski, A M, In Burtis C A, Ashwood, E R, eds. Tietz Textbook of Clinical Chemistry, 3^{rd} ed., Philadelphia, Pa.: WB Saunders: 1999:1601-1641; Manni, A., (1985), J. Clin. Endocrinol. Metab. 61(4):705-710; Robinson, S., (1 992), Br. J. Obstet. Gynecol. 99(3):232-238; Ooi, D S, (1999), Clin, Chem, 45(5):715). A commonly used method for determining bioavailable testosterone involves the selective precipitation of SHBG with ammonium sulfate. Similar to the assays for free testosterone described above, tritiated testosterone is added to serum, which is then allowed to come to equilibrium at physiologic temperature. Testosterone that is bound to SHBG is then selectively precipitated with 50% ammonium sulfate, which leaves free and albumin-bound testosterone in solution. The percentage of tritiated label that is not bound to SHBG is multiplied by the total testosterone to produce the bioavailable testosterone. Alternatively, the concentration of bioavailable testosterone can be measured directly by radioimmunoassay in the supernatant after solvent extraction (Cheng, R W, (1986), Clin. Chem. 32(7):1411. Another technique for measuring bioavailable testosterone involves saturating SHBG binding sites with DHT. SHBG has a significantly stronger affinity for DHT than for testosterone. Addition of excess DHT to the sample effectively forces all the SHBG-bound testosterone into solution. The non-protein-bound fraction is then measured after equilibrium dialysis or centrifugal ultrafiltration (Loric, S. (1988), Clin. Chem., 34(9):1826-1829).

Salivary Testosterone

Using salivary samples for the estimation of plasma-free testosterone levels is an attractive concept because of the ease of sample collection. In general, steroid levels in saliva are thought to reflect the free levels in the blood (Wheeler, M J, (1995), Ann. Clin. Biochem. 32(4):345-357; Gronowski, A M, In Burtis C A, Ashwood, E R, eds. Tietz Textbook of Clinical Chemistry, 3^{rd} ed., Philadelphia, Pa.: WB Saunders: 1999:1601-1641; Pearce, S., (1989), Clin. Chem. 35(4):632-635). Despite the fact that a number of laboratories offer salivary testosterone testing, this methodology has not gained widespread acceptance for routine clinical applications. Salivary testosterone levels are very low, especially in women. Currently available salivary testosterone methods have been effectively used in studies where ease of sample collection is a priority and the mean testosterone levels of large populations are compared. However, salivary testosterone methods have not been shown to be sensitive enough to produce diagnostically accurate results for the clinical assessment of individual patients, especially women. Salivary testosterone measurement may play a more significant role in the future as more sensitive techniques are developed and appropriately validated. Ultimately, the clinical utility of salivary testosterone measurement will depend on its analytical correlation with other, more established assays of testosterone status.

The more recent diagnostic assay kits now available for measuring testosterone levels include, for example, the following: Oxford Biomedical Research provides a Testosterone EIA kit. Biocompare also manufactures an 11-keto testosterone EIA kit. Immunometrics also manufactures a testosterone competitive EIA kit for measuring serum or plasma testosterone. The Bayer Immuno 1 Testosterone assay (Bayer Corporation) has also been used successfully to provide an accurate assessment of testosterone levels in clinical specimens (Levesque, A. (1998), Clin. Biochem. 31(1):23-28). In addition, R & D Systems also manufactures a testosterone ELISA kit (catalog No. DE2300).

The purpose of the studies described herein was to examine the relationship between total serum testosterone level and serum PSA, PSAD, cPSA, cPSAD, prostate volume, and prostate cancer diagnosis in patients presenting for prostate biopsy. In addition, a formula has been derived to adjust PSAD for serum testosterone in order to improve the specificity of PSAD in prostate cancer detection. A normal range of values for testosterone corrected PSA or testosterone corrected PSAD or testosterone corrected PSAD-TZ (transition zone) may be determined and established using standard methods of measuring PSA, testosterone and prostate volume in normal patients. Once these values are obtained and established for normal patients, one of skill in the art would recognize whether a sample taken from a patient suspected of having prostate cancer or who is at risk of developing prostate cancer falls within the normal range or outside of the normal range.

Immunodetection Assays

The present invention utilizes immunodetection methods for binding, purifying, removing, quantifying or otherwise generally detecting biological components. The encoded proteins or peptides or hormones of the present invention, for example, PSA and testosterone, may be detected by antibodies having reactivity therewith, or, alternatively, antibodies prepared in accordance with general procedures known to those skilled in the art, may be employed to detect the encoded proteins, peptides or hormones.

In general, the immunobinding methods include obtaining a sample suspected of containing a protein, peptide, hormone or antibody, and contacting the sample with an antibody or protein, peptide or hormone in accordance with the present invention, as the case may be, under conditions effective to allow the formation of immunocomplexes.

The immunobinding methods include methods for detecting or quantifying the amount of a reactive component in a sample, which methods require the detection or quantitation of any immune complexes formed during the binding process. Here, one would obtain a sample suspected of containing a prostate disease-marker encoded protein, peptide or a corresponding antibody, and contact the sample with an antibody or encoded protein or peptide, as the case may be, and then detect or quantify the amount of immune complexes formed under the specific conditions.

In terms of antigen detection, the biological sample analyzed may be any sample that is suspected of containing a prostate cancer-specific antigen, such as a prostate or lymph node tissue section or specimen, a homogenized tissue extract an isolated cell, a cell membrane preparation, separated or purified forms of any of the above protein-containing compositions, or even any biological fluid that comes into contact with prostate tissues, including blood, lymphatic fluid, and even seminal fluid.

Contacting the chosen biological sample with the protein, peptide or antibody under conditions effective and for a period of time sufficient to allow the formation of immune complexes (primary immune complexes) is generally a matter of simply adding the composition to the sample and incubating the mixture for a period of time long enough for the antibodies to form immune complexes with, i.e., to bind to, any antigens present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any radioactive, fluorescent, biological or enzymatic tags or labels of standard use in the art. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody or a biotin/avidin ligand binding arrangement, as is known in the art.

The encoded protein, peptide or corresponding antibody employed in the detection may itself be linked to a detectable label, wherein one would then simply detect this label, thereby allowing the amount of the primary immune complexes in the composition to be determined.

Alternatively, the first added component that becomes bound within the primary immune complexes may be detected by means of a second binding ligand that has binding affinity for the encoded protein, peptide or corresponding antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are then generally washed to remove any non-specifically bound labelled secondary antibodies or ligands, and the remaining label in the secondary immune complexes is then detected.

Further methods include the detection of primary immune complexes by a two step approach. A second binding ligand, such as an antibody, that has binding affinity for the encoded protein, peptide or corresponding antibody is used to form secondary immune complexes, as described above. After washing, the secondary immune complexes are contacted with a third binding ligand or antibody that has binding affinity for the second antibody, again under conditions effective and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

The immunodetection methods of the present invention have evident utility in the diagnosis of conditions such as prostate cancer and benign prostate hyperplasia. Here, a biological or clinical sample suspected of containing either the encoded protein or peptide or corresponding antibody is used.

In the clinical diagnosis or monitoring of patients with prostate cancer, the detection of an antigen encoded by a prostate cancer marker nucleic acid, or an increase in the levels of such an antigen, in comparison to the levels in a corresponding biological sample from a normal subject is indicative of a patient with prostate cancer. The basis for such diagnostic methods lies, in part, with the finding that the nucleic acid prostate cancer markers identified in the present invention are overexpressed in prostate cancer tissue samples. By extension, it may be inferred that at least some of these markers produce elevated levels of encoded proteins, that may also be used as prostate cancer markers.

Those of skill in the art are very familiar with differentiating between significant expression of a biomarker, which represents a positive identification, and low level or background expression of a biomarker. Indeed, background expression levels are often used to form a “cut-off” above which increased staining will be scored as significant or positive. Significant expression may be represented by high levels of antigens in tissues or within body fluids, or alternatively, by a high proportion of cells from within a tissue that each give a positive signal.

Immunohistochemistry

The antibodies specific for the proteins of the present invention, including PSA or testosterone, may be used in conjunction with both fresh-frozen and formalin-fixed, paraffin-embedded tissue blocks prepared by immunohistochemistry (IHC). Any IHC method well known in the art may be used such as those described in Diagnostic Immunopathology, 2nd edition edited by, Robert B. Colvin, Atul K. Bhan and Robert T. McCluskey. Raven Press, N.Y., 1995, (incorporated herein by reference) and in particular, Chapter 31 of that reference entitled Gynecological and Genitourinary Tumors (ages 579-597), by Debra A Bell, Robert H. Young and Robert E. Scully and references therein.

ELISA Assays

As noted, it is contemplated that the proteins or peptides or hormones of the invention may find utility as immunogens, e.g., for preparing antibodies for use in immunohistochemistry and in ELISA assays. One evident utility of the encoded antigens and corresponding antibodies is in immunoassays for the detection of prostate disease marker proteins, as needed in diagnosis and prognostic monitoring.

Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and Western blotting, dot blotting, FACS analyses, and the like may also be used.

In one exemplary ELISA, antibodies binding to the proteins of the invention are immobilized onto a selected surface exhibiting protein affinity, such as a well in a polystyrene microtiter plate. Then, a test composition suspected of containing the prostate disease marker antigen, such as a clinical sample, is added to the wells. After binding and washing to remove non-specifically bound immune complexes, the bound antigen may be detected. Detection is generally achieved by the addition of a second antibody specific for the target protein that is linked to a detectable label. This type of ELISA is a simple “sandwich ELISA”. Detection may also be achieved by the addition of a second antibody, followed by the addition of a third antibody that has binding affinity for the second antibody, with the third antibody being linked to a detectable label.

In another exemplary ELISA, the samples suspected of containing the prostate disease marker antigen are immobilized onto the well surface and then contacted with the antibodies of the invention. After binding and washing to remove non-specifically bound immune complexes, the bound antigen is detected. Where the initial antibodies are linked to a detectable label, the immune complexes may be detected directly. Again, the immune complexes may be detected using a second antibody that has binding affinity for the first antibody, with the second antibody being linked to a detectable label.

Another ELISA in which the proteins or peptides are immobilized, involves the use of antibody competition in the detection. In this ELISA, labelled antibodies are added to the wells, allowed to bind to the prostate disease marker protein, and detected by means of their label. The amount of marker antigen in an unknown sample is then determined by mixing the sample with the labelled antibodies before or during incubation with coated wells. The presence of marker antigen in the sample acts to reduce the amount of antibody available for binding to the well and thus reduces the ultimate signal. This is appropriate for detecting antibodies in an unknown sample, where the unlabeled antibodies bind to the antigen-coated wells and also reduces the amount of antigen available to bind the labeled antibodies.

Irrespective of the format employed, ELISAs have certain features in common, such as coating, incubating or binding, washing to remove non-specifically bound species, and detecting the bound immune complexes. These are described as follows:

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the control human prostate cancer and/or clinical or biological sample to be tested under conditions effective to allow immune complex (antigen/antibody) formation. Detection of the immune complex then requires a labeled secondary binding ligand or antibody, or a secondary binding ligand or antibody in conjunction with a labeled tertiary antibody or third binding ligand. “Under conditions effective to allow immune complex (antigen/antibody) formation” means that the conditions preferably include diluting the antigens and antibodies with solutions such as BSA, bovine gamma globulin (BGG) and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 hours, at temperatures preferably on the order of 25° to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact and incubate the first or second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azido-di-(3-ethyl-benzthiazoline-6-sulfonic acid >ABTS! and H₂O₂, in the case of peroxidase as the enzyme label. Quantitation is then achieved by measuring the degree of color generation, e.g., using a visible spectra spectrophotometer.

Use of Antibodies for Radioimaging

The antibodies of this invention specific for PSA and/or testosterone will be used to quantify and localize the expression of these proteins. The antibody, for example, will be labeled by any one of a variety of methods and used to visualize the localized concentration of the PSA and/or testosterone in patient samples.

The invention also relates to an in vivo method of imaging a pathological prostate condition using the above described monoclonal antibodies. Specifically, this method involves administering to a subject an imaging-effective amount of a detectably-labeled prostate cancer-specific monoclonal antibody or fragment thereof and a pharmaceutically effective carrier and detecting the binding of the labeled monoclonal antibody to the diseased tissue. The term “in vivo imaging” refers to any method which permits the detection of a labeled monoclonal antibody of the present invention or fragment thereof that specifically binds to a diseased tissue located in the subject's body. A “subject” is a mammal, preferably a human. An “imaging effective amount” means that the amount of the detectably-labeled monoclonal antibody, or fragment thereof administered is sufficient to enable detection of binding of the monoclonal antibody or fragment thereof to the diseased tissue.

A factor to consider in selecting a radionuclide for in vivo diagnosis is that the half-life of a nuclide be long enough so that it is still detectable at the time of maximum uptake by the target, but short enough so that deleterious radiation upon the host, as well as background, is minimized. Ideally, a radionuclide used for in vivo imaging will lack a particulate emission, but produce a large number of photons in a 140-2000 keV range, which may be readily detected by conventional gamma cameras.

A radionuclide may be bound to an antibody either directly or indirectly by using an intermediary functional group. Intermediary functional groups which are often used to bind radioisotopes which exist as metallic ions to antibody are diethylenetriaminepentaacetic acid (DTPA) and ethylene diaminetetracetic acid (EDTA). Examples of metallic ions suitable for use in this invention are ⁹⁹Tc, ¹²³I, ¹³¹I, ¹¹¹In, ¹³¹I, ⁹⁷Ru, ⁶⁷Cu, ⁶⁷Ga, ¹²⁵I, ⁶⁸Ga, ⁷²As, ⁸⁹Zr, and ²⁰¹Tl.

In accordance with this invention, the monoclonal antibody or fragment thereof specific for PSA or testosterone may be labeled by any of several techniques known to the art. The methods of the present invention may also use paramagnetic isotopes for purposes of in vivo detection. Elements particularly useful in Magnetic Resonance Imaging (“MRI”) include ¹⁵⁷Gd, ⁵⁵Mn, ¹⁶²Dy, ⁵²Cr, and ⁵⁶Fe.

Administration of the labeled antibody may be local or systemic and accomplished intravenously, intraarterially, via the spinal fluid or the like. Administration may also be intradermal or intracavitary, depending upon the body site under examination. After a sufficient time has lapsed for the monoclonal antibody or fragment thereof to bind with the diseased tissue, for example 30 minutes to 48 hours, the area of the subject under investigation is examined by routine imaging techniques such as MRI, SPECT, planar scintillation imaging and emerging imaging techniques, as well. The exact protocol will necessarily vary depending upon factors specific to the patient, as noted above, and depending upon the body site under examination, method of administration and type of label used; the determination of specific procedures would be routine to the skilled artisan. The distribution of the bound radioactive isotope and its increase or decrease with time is then monitored and recorded. By comparing the results with data obtained from studies of clinically normal individuals, the presence and extent of the diseased tissue may be determined.

It will be apparent to those of skill in the art that a similar approach may be used to radio-image the production of the encoded prostate disease marker proteins in human patients. The present invention provides methods for the both in vitro and in vivo diagnosis of prostate cancer in a patient. Such methods generally comprise administering to a patient an effective amount of a prostate cancer specific antibody, which antibody is conjugated to a marker, such as a radioactive isotope or a spin-labeled molecule, which is detectable by non-invasive methods. Alternatively, a patient sample is obtained, such as a sample of prostatic tissue from a biopsy sample or a body fluid such as whole blood, serum or plasma and these samples are used for measurement of PSA and testosterone. For the in vivo assessment, the antibody-marker conjugate is allowed sufficient time to come into contact with reactive antigens that be present within the tissues of the patient, and the patient is then exposed to a detection device to identify the detectable marker.

Measurement of Prostate Volume

There are various methods that may be used to determine prostate volume. It may be measured by digital rectal examination, cystourethrography, or urethrocystoscopy, but all of these are known to be inaccurate (Jensen K M E, Bruskewitz R C, Iversen P, Madsen P O, (1983), Urologia Internationalis, 33:173-178; Meyhoff H H, Hald T, (1978), Scand J Urol Nephrol;12:219-221; Meyhoff H H, Ingemann L, Nordling J, Hald T, (1981), Scand J Urol Nephrol, 15:45-51). For this reason, ultrasound scanning has gained wide popularity in the past few years (Wadanabe H, Igari D, Tanahashi Y, Harada K, Saitoh M. (1974), Tohoku J Exp Med,114:277-285; Abu-Yousef M M, Narayana A S, (1982), JCU, 10:275-278; Walz P H, Wen Deroth U, Jacobi G H (1983), Eur Urol, 9:148-152; Smith H J, Haveland H, (1982), Br J Urol, 54:531-535; Henneberry M, Carter M F, Neiman H I, (1979), J Urol, 12:615-616; Bartsch G, Egender G, Huebscher H, Rohr H (1982), J Urol,127:1119-1121; Hastak S M, Gammelgaard J, Holm H H (1982), J Urol, 127:1115-1118). Three different ultrasound approaches are available: the transrectal, the transurethral, and the transabdominal, though prostate volume measurement using the transrectal approach appears to be most accurate (Wadanabe H, Igari D, Tanahashi Y, Harada K, Saitoh M. (1974), Tohoku J Exp Med, 114:277-285; Hastak S M, Gammelgaard J, Holm H H. (1982), J Urol, 127:1115-1118).

Three commonly used prostate volume measurement techniques in transrectal ultrasonography (TRUS) are planimetry calculation, prolate ellipse volume calculation, and an ellipsoid volume measurement technique. For example, Kimura et al. described a biplane planimetry method for calculating prostatic volume (Kimura A. et al. (1997) J. Med. Ultrasound 5 (Suppl):31-34; Kimura, A. et al. (1997), Int. J. Urol 4(2): 152-156). Using this method, the contours of the prostate are traced using both cross and sagittal sections. Based on both the cross and sagittal contours, a non-ellipsoidal model is created. The model is composed of sequentially arranged copies of the cross section, which are reduced so that the anteroposterior diameters (height; H) of the copies fit the contour of the sagittal section. The areas of the copies are reduced in proportion with the square of the reduced rates of the height (H2), and so the formula for biplane planimetry is given as: 1×Amax×f°(Hi/Hmax)² where 1 is a stepped interval of the arrangement of copies, Amax is the area of the maximum cross section, Hmax is the height of the maximum cross section, and Hi are the heights measured at certain intervals in the sagittal section where the reduced copies are arranged. On the other hand, prolate ellipse volume calculation is determined as follows: prolate ellipse volume (centimeters)=(height×length×width)×Pi/6. Prostate volume in cubic centimeters as calculated by the ellipsoid volume method is determined by using the formula: height (H)×width (W)×length (L)×0.52. Transverse diameter (width) is defined as the maximal transverse diameter at mid-gland level, while longitudinal diameter (length) is defined as the distance from the proximal external sphincter to the urinary bladder (Litttrup P J, Williams C R, Egglin T K, Kane R A. (1991), Radiology, 179:49-53). Anteroposterior diameter (height) may be measured in two planes-axial and sagittal. Most authors have employed midsagittal scanning, but some have measured the diameter perpendicular to the transverse diameter seen on transaxial scans (Gerald J. Matthews, Joseph Motta, John A. Fracchia. (1996), J Clin Ultrasound, 24:501-505; Sung, B P et al. (2000), Korean J. Radiol. 1(2):110-113). Other means of measuring prostate volume include computerized tomography (CT) or magnetic resonance imaging (MRI) techniques.

Assessment of Prostate Tumor Cell Aggressiveness/Invasiveness

In order to assess the likelihood or potential for a prostate tumor to metastasize in an individual diagnosed with prostate cancer, the Gleason score is used by most physicians to aid in this assessment. The “Gleason score or Gleason grade” refers to a means for grading prostate cancer and relates to the degree of aggressiveness of the tumor. This grade imparts a significant correlation to the potential prognosis and is an important factor in recommending a particular therapy for that patient. The Gleason system is based on the architectural pattern of the glands of the prostate tumor. A tumor whose structure is nearly normal (well differentiated) will probably have a biological behavior relatively close to normal—not very aggressively malignant. The Gleason grade and score are key pieces of information for making treatment decisions.

Gleason Grades 1 and 2: These two grades closely resemble normal prostate. They are the least important grades because they seldom occur in the general population and because they confer a prognostic benefit which is only slightly better than grade 3. Both of these grades are composed by very pale glands which grow closely together. In grade 1 they form a compact mass; in grade 2 they are more loosely aggregated, and some glands wander (invade) into the surrounding muscle (stroma).

Gleason Grade 3: This is the most common grade by far and is also considered well differentiated (like grades 1 and 2). This is because all three grades have a normal “gland unit” like that of a normal prostate; that is, every cell is part of a circular row which forms the lining of a central space (the lumen). The lumen contains prostatic secretion like normal prostate, and each gland unit is surrounded by prostate muscle which keeps the gland units apart. In contrast to grade 2, wandering of glands (invading) into the stroma (muscle) is very prominent and is the main defining feature. The cells are dark rather than pale and the glands often have more variable shapes.

Gleason Grade 4: This is probably the most important grade because it is fairly common and because of the fact that if a lot of it is present, patient prognosis is usually (but not always) worsened by a considerable degree. Here also there is a big jump in loss of architecture. For the first time, we see disruption and loss of the normal gland unit. In fact, grade 4 is identified almost entirely by loss of the ability to form individual, separate gland units, each with its separate lumen.

Gleason Grade 5: Gleason grade 5 is an important grade because it usually predicts another significant step towards poor prognosis. Its overall importance for the general population is reduced by the fact that it is less common than grade 4, and it is seldom seen in men whose prostate cancer is diagnosed early in its development. This grade too shows a variety of patterns, all of which demonstrate no evidence of any attempt to form gland units. Although never an absolute the results with any form of conventional therapy is poor with this category.

The “Combined Gleason Score or Gleason Sum” can be explained as follows. When a pathologist looks at prostate cancer specimens under the microscope and gives them a Gleason Grade, he or she in fact will always try to identify two architectural patterns and assign a Gleason Grade to each one. There may be a primary pattern and then a secondary pattern which the pathologist will seek to describe for each specimen; alternatively, there may often be only a single pure grade.

In developing his system, Dr. Gleason discovered that by giving a combination of the grades of the two most common patterns he could see in any particular patient's specimens, he was better able to predict the likelihood that that particular patient would do well or badly. Therefore, even though it may seem confusing, the Gleason score which a physician usually gives to a patient is actually a combination or sum of two numbers. These combined Gleason sums or scores may be determined as follows:

The lowest possible Gleason score is 2 (1+1), where both the primary and secondary patterns have a Gleason Grade of 1 and therefore when added together their combined sum is 2. Very typical Gleason scores might be 5 (2+3), where the primary pattern has a Gleason grade of 2 and the secondary pattern has a grade of 3, or 6 (3+3), a pure pattern. Another typical Gleason score might be 7 (4+3), where the primary pattern has a Gleason grade of 4 and the secondary pattern has a grade of 3.

Finally, the highest possible Gleason score is 10 (5+5), when the primary and secondary patterns both have the most disordered Gleason grades of 5.

The grade of a prostate cancer specimen is very valuable to doctors in helping them to understand how a particular case of prostate cancer can be treated. In general, the time for which a patient is likely to survive following a diagnosis of prostate cancer is related to the Gleason score. The lower the Gleason score, the better the patient is likely to do. However, remember that prostate cancer is a very complicated disease. People with low Gleason scores have been known to fare poorly and men with high Gleason scores have been known to do well. General principles do not always apply to individual patients.

Kits

In still further embodiments, the present invention concerns immunodetection kits for use with the immunodetection methods described above. Furthermore, such measurements of PSA and testosterone, when combined with determination of prostate volume using any of the methods described herein, will aid in the assessment of the patient's status regarding the presence or absence of a prostate tumor, or for assessing a patient's risk of developing a prostate tumor. It may be envisioned that such kits will contain the reagents to perform both PSA and testosterone measurements concurrently so that when both values are determined, they may be used with the prostate volume measurements obtained to provide an accurate means of determining a patients likelihood of having prostate cancer, or metastasis associated with such cancer, or a recurrence of said cancer, or to determine the aggressiveness of such cancer. The kits may also be used for pre-treatment staging of the prostate cancer or to assess the effectiveness of treatment of such cancer. As the PSA and testosterone specific antibodies may be employed to detect their specific antigens, either or both of such components may be provided in the kit. Moreover, a kit may be designed for measurement of at least one type of PSA, including any isoform of free or complexed PSA as well as free or total testosterone. However, it is also contemplated that the kit may include reagents to measure more than one type of PSA concurrently, including PSA, cPSA, B-PSA, pro-PSA, or HK2. These kits may also contain the reagents to measure free, bound or total testosterone. Thus, a kit is envisioned that contains the reagents to measure PSA and/or its multiple isoforms, as well as free or total testosterone, such that these values are obtained concurrently, and when combined with the prostate volume measurement, can be used to determine the testosterone corrected PSA density value. Depending on the assay itself (competitive or non-competitive ELISA or RIA) the immunodetection kits will thus comprise, in suitable container means, the proteins, peptides, hormones or the first and/or second antibodies that bind to the protein, peptide, or hormone and an immunodetection reagent.

In a particular embodiment, testosterone and PSA or isoforms of PSA may be quantitated using standard reagents and kits, which are commercially available to measure each marker individually. In another embodiment, testosterone and PSA or its isoforms are measured concurrently in the same kit. Such a kit would contain reagents specific for each of the analytes. Thus, the present invention provides a more quantitative and accurate means of assessing a subject's risk for developing prostate cancer, or for pre-treatment staging of prostate cancer, or for monitoring for cancer recurrence or for determining the aggressiveness of a cancer or for determining if a patient has prostate cancer by measuring all of these markers concurrently. To the inventor's knowledge, no other art currently exists which describes combining the concurrent non-invasive techniques and measurements described herein for determining the presence of prostate cancer or for assessing a subject's risk for developing prostate cancer or for pre-treatment staging of prostate cancer.

In certain embodiments, the protein, peptide, hormone or the first antibody that binds to the protein, peptide, or hormone may be bound to a solid support, such as a column matrix or well of a microtiter plate.

The immunodetection reagents of the kit may take any one of a variety of forms, including those detectable labels that are associated with or linked to the given antibody or antigen, and detectable labels that are associated with or attached to a secondary binding ligand. Exemplary secondary ligands are those secondary antibodies that have binding affinity for the first antibody or antigen, and secondary antibodies that have binding affinity for a human antibody.

Further suitable immunodetection reagents for use in the present kits include the two-component reagent that comprises a secondary antibody that has binding affinity for the first antibody or antigen, along with a third antibody that has binding affinity for the second antibody, the third antibody being linked to a detectable label.

The kits may further comprise a suitably aliquoted composition of the protein, polypeptide or hormone antigen, whether labeled or unlabeled, as may be used to prepare a standard curve for a detection assay.

The kits may contain antibody-label conjugates either in fully conjugated form, in the form of intermediates, or as separate moieties to be conjugated by the user of the kit. The components of the kits may be packaged either in aqueous media or in lyophilized form.

The container means of the kits will generally include at least one vial, test tube, flask, bottle, syringe or other container means, into which the antibody or antigen may be placed, and preferably, suitably aliquoted. Where a second or third binding ligand or additional component is provided, the kit will also generally contain a second, third or other additional container into which this ligand or component may be placed. The kits of the present invention will also typically include a means for containing the antibody, antigen, and any other reagent containers in close confinement for commercial sale. Such containers may include injection or blow-molded plastic containers into which the desired vials are retained.

While the kits described above provide the accuracy and sensitivity necessary for measurements of testosterone and PSA as described in the present invention, further kits may be developed that contain the antibodies, reagents, buffers, standards and instructions for assaying both markers using the same format, e.g. ELISA, or a calorimetric assay. The test kits would be modified appropriately depending on whether the samples to be assayed consist of whole cells, cell lysates, plasma, serum, urine or saliva or a combination thereof. Thus, at least two markers that are used for detection of prostate cancer may be measured concurrently using the same assay format.

Thus, an assay format is preferred in which binding partners such as antibodies can be obtained or prepared for the analytes (testosterone and PSA). Biotin-avidin, biotin-streptavidin or other biotin-binding-reagent reactions can be used to enhance or modulate the test. However, any such assay can be devised using other binding partners to the analytes, including but not limited to extracellular or intracellular receptor proteins which recognize the analytes, binding fragments thereof, hybridization probes for nucleic acids, lectins for carbohydrates, etc. The particular selection of binding partners is not limiting, provided that the binding partners permit the test to operate as described herein. The preselected analytes, when present, are detectable by binding two binding partners, one immobilized on the test strip, or microtiter plate or whatever format the assay is provided) and another part of a conjugate. This is taken into consideration in the selection of the reagents for the assay.

If a dry test strip is desired, this may be set up in any format in which contact of the sample with the reagents is permitted and the formation and mobility of the immunocomplexes and other complexes forming therein are permitted to flow and contact an immobilized reagent at the capture line. Various formats are available to achieve this purpose, which may be selected by the skilled artisan.

The label portion of the mobile, labeled antibody to the marker may be a visible label, such as gold or latex, an ultraviolet absorptive marker, fluorescent marker, radionuclide or radioisotope-containing marker, an enzymatic marker, or any other detectable label. A visibly detectable marker or one that can be easily read in a reflectometer is preferred, for use by eye, reading or confirmation with a reflectometer. Other labels may be applicable to other semi-automated or automated instrumentation.

The conjugates of the invention may be prepared by conventional methods, such as by activation of an active moiety, use of homobifunctional or heterobifunctional cross-linking reagents, carbodiimides, and others known in the art. Preparation of, for example, a gold-labeled antibody, a conjugate between an antibody and an analyte (not an immunocomplex but a covalent attachment which allows each member to independently exhibit its binding properties), biotinylation of an antibody, conjugation of streptavidin with a protein, immobilization of antibodies on membrane surfaces, etc., are all methods known to one of skill in the art.

A kit may have at least one reagent for carrying out an assay of the invention, such as a kit comprising a conjugate between a biotin-binding reagent and an antibody to testosterone or PSA. Preferably, the kit comprises all of the reagents needed to carry out any one of the aforementioned assays, whether it be homogeneous, heterogeneous, comprise a single conjugate of the marker conjugated to an antibody to the analyte, or comprise two reagents which serve this function (such as a biotinylated antibody to the analyte plus a streptavidin-marker conjugate, or a biotinylated marker plus a streptavidin conjugated to an antibody to the analyte conjugate), or whether the assay employs an immobilized antibody to the analyte and a labeled antibody to a different site on the analyte. Referring to the first analyte as analyte and the second analyte as marker, and a second binding partner as a binding partner which recognizes a different epitope than the first binding partner mentioned, the kits are non-limiting examples of those embraced herein.

In the foregoing kits, the binding partners are preferably antibodies or binding portions thereof, and both the binding partner to the analytes (testosterone and PSA) and the second binding partner to the analytes capable of simultaneously binding to the analyte. The immobilized binding partner may be provided in the form of a capture line on a test strip, or it may be in the form of a microplate well surface or plastic bead. The kits may be used in a homogeneous format, wherein all reagents are added to the sample simultaneously and no washing step is required for a readout, or the kits may be used in a multi-step procedure where successive additions or steps are carried out, with the immobilized reagent added last, with an optional washing step.

The antibodies specific for the two markers may be obtained commercially, or can be produced by techniques known to those skilled in the art.

### EXAMPLES

The following examples are put forth so as to provide those of ordinary skill in the art with a complete disclosure and description of how to assess the levels of amyloid beta in a population of clinically depressed patients, and are not intended to limit the scope of what the inventors regard as their invention.

Methods

Frozen serum samples from 191 patients presenting for prostate biopsy were selected from a prospectively enrolled Institutional Review Board approved longitudinal serum bank, which included patients presenting to the Departments of Urology at a university hospital and a VA hospital between November 2001 and June 2003. Indications for prostate biopsy were 1) elevated PSA in 156 of 191 patients (81.7%) and 2) abnormal DRE in 35 of 191 patients (18.3%). Transrectal ultrasound (TRUS) prostate volume measurements were available for all 191 patients. The diagnosis of prostate cancer was established with systematic 12-core TRUS-guided biopsies from the peripheral zone of the prostate, including far lateral sampling. Of the patients with newly diagnosed prostate cancer, 73% were clinical stage T1c. Prior to serum banking, all patients were consented for procurement and banking of serum using a global consent approved by the Institutional Review Board. For serum banking, two vials of blood were collected by a designated procurement technician. Following formation of blood clot, serum was separated by centrifugation and aliquoted into 4-5 one mL cryotubes for storage at −80° C. In order to protect patient confidentiality, specimens were linked to a pre-existing clinical database by accession number only. Baseline demographic and clinical data from all patients included in the serum bank were compiled into a database utilizing Filemaker Pro (version 6.0) managed by two data managers. All outpatient office charts were reviewed by two data managers uninvolved in the care of the patients.

All serum samples were assayed for total serum testosterone, using a Bayer competitive magnetic separation immunoassay (nl range 165-830 ng/dL) (Bayer Diagnostics, Tarrytown, N.Y.), and for PSA and cPSA, using the Bayer Immuno 1 tPSA and cPSA assay, which is a heterogeneous sandwich magnetic separation immunoassay (Bayer Diagnostics, Tarrytown, N.Y.). For each sample, testing for testosterone, PSA, and cPSA was performed within the same freeze-thaw cycle. Samples were tested in singlets. In order to confirm the accuracy of the testosterone test, 63 patients had a repeat testosterone level drawn after a negative prostate biopsy.

The independent variables were serum testosterone (T), gonadal status, and prostate cancer diagnosis. Patients were divided into 2 gonadal status subgroups: hypogonadal (T<300) and eugonadal (T≧300). Patients were also stratified into 2 prostate cancer diagnosis subgroups: prostate cancer and no prostate cancer.

The dependent variables were age, serum PSA, PSAD, T-corrected PSAD, cPSA, cPSAD, T-corrected PSAD, prostate volume, and prostate cancer diagnosis. PSAD and cPSAD were calculated using the formula (PSA/prostate volume). T-corrected PSAD and cPSAD were calculated using the formula (PSA/(prostate volume×testosterone)). Prostate volume was based on transrectal ultrasound measurement (TRUS) measurements, which were taken in the greatest dimension. Prostate volume was calculated by the ellipsoid volume method of H×W×L×0.52. The diagnosis of prostate cancer was analyzed by calculating the percentage of patients diagnosed with prostate cancer.

Differences between gonadal status subgroups for each dependent variable were analyzed for statistical significance using analysis of variance (one-way Anova). All statistical tests were performed using a criterion of p=0.05 as evidence of statistical significance. Statistical analyses were performed using JMP statistical software (version 4.0.4) developed by SAS Institute (Cary, N.C.).

Results

The baseline characteristics of 191 patients presenting for prostate biopsy are summarized in Table 1. The group of patients was stratified into two subgroups based upon gonadal status: 40 hypogonadal (T<300) patients (20.9%) and 151 eugonadal (T≧300) patients (79.1%). The baseline characteristics of each gonadal status subgroup and the comparison between the subgroups are also summarized in Table 1. There were no statistically significant differences in age, PSA, PSAD, cPSA, and cPSAD between the gonadal status subgroups. There were statistically significant differences in T-corrected PSAD (p<0.0001) and T-corrected cPSAD (p<0.0001), with the T-corrected values higher in the hypogonadal subgroup. There was a statistically significant difference in prostate volume (p=0.01) between the gonadal status subgroups. Prostate volume was highest in the hypogonadal subgroup and lowest in the eugonadal subgroup. In addition, there was an increase in the percentage of men diagnosed with prostate cancer in the hypogonadal subgroup (32.5%) compared to the eugonadal subgroup (24.5%). However, this was not statistically significant.

The group of patients was also stratified into two subgroups based upon prostate cancer diagnosis: 141 patients without prostate cancer (73.8%) and 50 patients with prostate cancer (26.2%). The baseline characteristics of each prostate cancer diagnosis subgroup and the comparison between the subgroups are summarized in Table 2. There were no statistically significant differences in age, testosterone, PSA, cPSA, and cPSAD between the prostate cancer diagnosis subgroups. There was a statistically significant difference in PSAD (p=0.045), with PSAD higher in the prostate cancer subgroup. There were statistically significant differences in T-corrected PSAD (p=0.014) and T-corrected cPSAD (p=0.022), with the T-corrected values higher in the prostate cancer subgroup. There was no statistically significant difference in prostate volume between the prostate cancer diagnosis subgroups.

Table 3 summarizes data correlating serum testosterone with age, PSA, PSAD, T-corrected PSAD, cPSA, cPSAD, T-corrected cPSAD, prostate volume, and prostate cancer diagnosis.

Age, PSA, cPSA, PSAD, cPSAD, T-corrected PSAD, and T-corrected cPSAD were evaluable for all 191 patients. Age was not significantly related to testosterone in the total group of patients or in either of the gonadal status subgroups. PSA, PSAD, cPSA, and cPSAD were not significantly related to testosterone in the total group of patients or in either of the gonadal status subgroups. There was a negative correlation between testosterone and T-corrected PSAD in the total group of patients (r=−0.312, p<0.0001) and in the eugonadal subgroup (r=−0.197, p=0.015), but no correlation in the hypogonadal subgroup. Likewise, there was a negative correlation between testosterone and T-corrected cPSAD in the total group of patients (r=−0.287, p<0.0001) and in the eugonadal subgroup (r=−0.173, p=0.033, but no correlation in the hypogonadal subgroup.

Prostate volume was evaluable for all 191 patients. There was a negative correlation between testosterone and prostate volume in the total group of patients (r=−0.209, p=0.004). However, prostate volume was not significantly related to testosterone in either of the gonadal status subgroups.

Diagnosis of prostate cancer was evaluable for all 191 patients. The percentage diagnosed with prostate cancer was 26.2%, 32.5%, and 24.5% for the total group of patients, hypogonadal subgroup, and eugonadal subgroup, respectively. Diagnosis of prostate cancer was not significantly related to testosterone in the total group of patients or in either of the gonadal status subgroups.

63 patients had a repeat testosterone level drawn after a negative prostate biopsy. There was a strongly positive correlation between this testosterone level and the initial testosterone level in these patients (r=0.626, p<0.0001), confirming the accuracy of the testosterone test.

Summary

Testosterone is produced by the Leydig cells of the testes when these cells are stimulated by luteinizing hormone (LH) produced by the pituitary gland. Testosterone production is controlled by the hypothalamic-pituitary-gonadal axis. In the prostate, the enzyme 5α-reductase converts testosterone into dihydrotestosterone (DHT), the principal androgen in the prostate and a more potent androgen than testosterone. 90% of total prostatic androgen is in the form of DHT, principally derived from testicular androgens (McConnell, J. D.: (1995), Br J Urol, 76 (Suppl 1): 5).

The suppression of androgen hormones has been found to reduce serum PSA. Gonadotropin-releasing hormone (GnRH) agonism (Weber, J. P., Oesterling, J. E., Peters, C. A. et al., (1989), J Urol, 141: 987) androgen receptor blockade (Stone, N. N. and Clejan, S. J., (1991) J Androl, 12: 376) and 5α-reductase inhibition (McConnell, J. D., Wilson, J. D., George, F. W. et al., (1992), J Clin Endocrinol Metab, 74: 505; Gormley, G. J., Ng, J., Cook, T. et al., (1994), Urology, 43: 53) have been found to decrease serum PSA levels by 46% to 82%. Based on these studies, there appears to be a direct relationship between serum PSA and serum testosterone when serum testosterone is reduced to castrate levels. In our study, no patient had a castrate level of testosterone, with the lowest level of testosterone being 136. One would expect a similar direct relationship between PSA and testosterone in hypogonadal men. However in our study, there were no statistically significant differences in either PSA or cPSA between the gonadal status subgroups (Table 1). In addition, there was no correlation between testosterone and either PSA or cPSA in the total group of patients or in either of the gonadal status subgroups (Table 3). Of note, the prevalence of hypogonadism found in our study was similar to the general population (Harman, S. M., Metter, E. J., Tobin, J. D. et al., (2001), J Clin Endocrinol Metab, 86: 724). Most investigators have found no correlation between total serum testosterone and serum PSA level (Monath, J. R., McCullough, D. L., Hart, L. J. et al., (1995), Urology, 46: 58; Aus, G., Bergdahl, S., Hugosson, J. et al., (1994), Scandinavian Journal of Urology & Nephrology, 28: 379; Hoffman, M. A., DeWolf, W. C. and Morgentaler, A., (2000), J Urol, 163: 824; Kubricht, W. S., Williams, B. J., Whatley T. et al., (1999), Urology, 54: 1035; Monda, J. M., Myers, R. P., Bostwick, D. G. et al., (1995), Urology, 46: 62; Sairam, K., Kulinskaya, E., Boustead, G. B. et al., (2002), BJU International, 89: 261; Schatzl, G., Reiter, W. J., Thurridl, T. et al., (2000), Prostate, 44: 219). Monath et al found that this was still true even when corrected for age and weight (Monath, J. R., McCullough, D. L., Hart, L. J. et al., (1995), Urology, 46: 58. Aus et al also found no correlation between testosterone and PSAD (Aus, G., Bergdahl, S., Hugosson, J. et al., (1994), Scandinavian Journal of Urology & Nephrology, 28: 379). Schatzl et al examined patients with newly diagnosed prostate cancer and found a trend toward lower PSA in hypogonadal men but this difference was not statistically significant (Schatzl, G., Madersbacher S., Thurridl, T. et al., (2001), Prostate, 47: 52). Significantly lower PSA levels in hypogonadal men were found by Behre et al and Guay et al (Behre, H. M., Bohmeyer, J. and Nieschlag, E., (1994), Clinical Endocrinology, 40: 341; Guay, A. T., Perez, J. B., Fitaihi, W. A. et al., (2000), Endocrine Practice, 6: 132).

A related question is that of the relationship between testosterone and prostate volume. A study by Lee et al described a bell-shaped growth response to androgen stimulation and a dose-dependent induction of PSA production in LNCaP cells (Lee, C., Sutkowski, D. M., Sensibar, J. A. et al., (1995), Endocrinology, 136: 796). The LNCaP cell line is an androgen-sensitive human prostatic cancer cell line. The addition of DHT to culture medium at low concentrations resulted in cellular proliferation in a dose-dependent manner. However, a further increase in DHT concentration resulted in a progressive decline in cellular proliferation. These observations may help explain our finding of a progressive decrease in prostate volume from hypogonadal (mean prostate volume 57.1±6.9 cc) to eugonadal (mean prostate volume 43.8±1.9 cc) subgroups (p=0.01) (Table 1). Similarly, a negative correlation was found between prostate volume and testosterone, which was statistically significant in the total group of patients (r=−0.209, p=0.004) and close to statistically significant in the eugonadal subgroup (r=−0.146, p=0.073) (Table 3). In a prospective study of 207 men presenting with clinical features of age-related androgen deficiency and elevated LH, Pechersky et al found that treating patients with oral testosterone undecanoate caused marked decreases in prostate volume and PSA (Pechersky, A. V., Mazurov, V. I., Semiglazov, V. F. et al., (2002), Int J Androl, 25: 119. This finding further supports our finding of a negative correlation between prostate volume and testosterone. The study presented here is the only one to document a progressive decrease in prostate volume from hypogonadal to eugonadal men. No correlation between total serum testosterone and prostate volume was found by Hoffman et al and Kubricht et al (Hoffman, M. A., DeWolf, W. C. and Morgentaler, A., (2000), J Urol, 163: 824; Kubricht, W. S., Williams, B. J., Whatley T. et al., (1999), Urology, 54: 1035). No correlation between total serum testosterone and pathologic prostate weight was found by Monda et al. (Monda, J. M., Myers, R. P., Bostwick, D. G. et al., (1995), Urology, 46: 62). In the study by Schatzl et al examining patients with newly diagnosed prostate cancer, no difference in prostate volume was found between hypogonadal and eugonadal men (Schatzl, G., Madersbacher S., Thurridl, T. et al., (2001), Prostate, 47: 52). Significantly lower prostate volumes were found in hypogonadal men by Behre et al. (Behre, H. M., Bohmeyer, J. and Nieschlag, E., (1994), Clinical Endocrinology, 40: 341). Joseph et al found that increasing levels of total serum testosterone were marginally associated with increasing prostate volume (p=0.058) in African American men (Joseph, M. A., Wei, J. T., Harlow, S. D. et al., (2002), Prostate, 53: 322).

A final question concerns the relationship between testosterone and diagnosis of prostate cancer. Carter et al evaluated serum testosterone levels in three age-matched groups of men who were part of the Baltimore Longitudinal Study of Aging: 16 men with no prostatic disease, 20 men with BPH, and 20 men with prostate cancer (Carter, H. B., Pearson, J. D., Metter, E. J. et al., (1995), Prostate, 27: 25). No significant difference in total testosterone was found among the groups at 0-5, 5-10, and 10-15 years before diagnosis, suggesting that there are no measurable differences in testosterone levels among men who are destined to develop prostate cancer and those without the disease. The studies presented here show an increase in the percentage of men diagnosed with prostate cancer in the hypogonadal subgroup (32.5%) compared to the eugonadal subgroup (24.5%), but this was not statistically significant (Table 1). Further supporting the findings of the study by Carter et al, when the patients here were stratified into cancer (mean testosterone 414.6±20.5 ng/dL)) and non-cancer (mean testosterone 435.0±12.8 ng/dL)) subgroups, no statistically significant difference in testosterone between the subgroups was found (Table 2).

No statistical difference in total serum testosterone levels between prostate cancer and non-prostate cancer patients was found by Kubricht et al, Schatzl et al, Carter et al, Andersson et al, and Morgentaler et al. (Kubricht, W. S., Williams, B. J., Whatley T. et al., (1999), Urology, 54: 1035; Schatzl, G., Reiter, W. J., Thurridl, T. et al., (2000), Prostate, 44: 219; Carter, H. B., Pearson, J. D., Metter, E. J. et al., (1995), Prostate, 27: 25; Andersson, S. O., Adami, H. O., Bergstrom, R. et al., (1993), British Journal of Cancer, 68: 97; Morgentaler, A., Bruning, C. O. and DeWolf, W. C., (1996), JAMA, 276: 1904. No correlation of total serum testosterone with risk of prostate cancer was found by Hoffman et al and Gann et al., (Hoffman, M. A., DeWolf, W. C. and Morgentaler, A., (2000), J Urol, 163: 824; Gann P. H., Hennekens, C. H., Ma, J. et al., (1996), Journal of National Cancer Institute, 88: 1118).

When patients were stratified into prostate cancer and non-prostate cancer subgroups, no statistically significant differences in PSA, cPSA, cPSAD, and prostate volume were found, however, a slightly statistically significant difference in PSAD (p=0.045) (Table 2) was found. Similarly, Morgentaler et al found no significant differences between cancer and non-prostate cancer subgroups with regard to PSA, PSAD, and prostate volume (Morgentaler, A., Bruning, C. O. and DeWolf, W. C., (1996), JAMA, 276: 1904.

A formula was derived to adjust PSAD for serum testosterone in order to improve the specificity of PSAD in prostate cancer detection, namely T-corrected PSAD. When patients were stratified into prostate cancer and non-prostate cancer subgroups, we found statistically significant differences in T-corrected PSAD (p=0.014) and T-corrected cPSAD (p=0.022) between the prostate cancer and non-prostate cancer subgroups, with the T-corrected values higher in the prostate cancer subgroup (Table 2). T-corrected PSAD and T-corrected cPSAD may prove to be useful in the diagnosis of prostate cancer.

Potential weaknesses of this study include the low number of patients in the hypogonadal subgroup and the measurement of total rather than bioavailable free serum testosterone. In addition, random serum testosterone rather than morning serum testosterone was measured. A future study will include a larger group of patients.

Conclusions

In the study presented here of men presenting for evaluation of possible prostate cancer, a surprisingly negative correlation was observed between total serum testosterone and prostate volume. Hypogonadal men had a statistically significantly higher prostate volume than did eugonadal men, despite comparable PSA, PSAD, cPSA, and cPSAD. No correlation was found between testosterone and PSA, PSAD, cPSA, and cPSAD. When we stratified patients into prostate cancer and non-prostate cancer subgroups, statistically significant differences were found in T-corrected PSAD and T-corrected cPSAD, with the T-corrected values higher in the prostate cancer subgroup. T-corrected PSAD and T-corrected cPSAD may prove to be useful in the diagnosis of prostate cancer. Hypogonadal men presenting for evaluation for prostate cancer have a trend toward increased risk of prostate cancer. However, diagnosis of prostate cancer was not significantly related to testosterone in the total group of patients or in either of the gonadal subgroups, and there was no statistically significant difference in testosterone between the cancer and non-cancer subgroups. When contemplating testosterone replacement in hypogonadal men with elevated PSA, prostate cancer should be aggressively excluded because of a potentially higher risk of prostate cancer than in eugonadal men.

TABLE 1
Baseline characteristics of study population and gonadal status
subgroups and comparison between gonadal status subgroups

| | | | Hypo- | Eu- | |
|---|---|---|---|---|---|
| | Total | | gonadal | gonadal | |
| | (n = 191) | Range | (n = 40) | (n = 151) | P value |
| Mean age ± SEM (yrs) | 65.1 ± 0.6 | 41-90 | 66.6 ± 1.3 | 64.6 ± 0.7 | 0.443 |
| Mean prostate | 46.6 ± 2.1 | 6.9-210.8 | 57.1 ± 6.9 | 43.8 ± 1.9 | 0.010 |
| volume ± SEM (cc) | | | | | |
| Mean PSA ± SEM (ng/mL) | 7.86 ± 0.42 | 0.88-49.02 | 7.86 ± 0.75 | 7.86 ± 0.49 | 0.998 |
| Mean PSAD ± SEM | 0.202 ± 0.012 | 0.019-1.358 | 0.193 ± 0.025 | 0.205 ± 0.014 | 0.688 |
| Mean T-corrected | 0.00052 ± 0.00003 | 0.00005-0.0033 | 0.00081 ± 0.00010 | 0.00045 ± 0.00003 | <0.0001 |
| PSAD ± SEM | | | | | |
| Mean cPSA ± SEM (ng/mL) | 6.00 ± 0.36 | 0.73-43.82 | 6.11 ± 0.67 | 5.97 ± 0.42 | 0.876 |
| Mean cPSAD ± SEM | 0.157 ± 0.010 | 0.013-1.209 | 0.154 ± 0.022 | 0.158 ± 0.012 | 0.898 |
| Mean T-corrected | 0.00041 ± 0.00003 | 0.00003-0.0030 | 0.00064 ± 0.00009 | 0.00034 ± 0.00003 | <0.0001 |
| cPSAD ± SEM | | | | | |
| Mean testosterone ± | 429.6 ± 10.8 | 136-944 | 239.1 ± 7.0 | 480.1 ± 10.2 | <0.0001 |
| SEM (ng/dL) | | | | | |
| % diagnosed with | 26.2 | | 32.5 | 24.5 | 0.314 |
| prostate cancer | | | | | |

TABLE 2
Baseline characteristics of study population and prostate cancer
subgroups and comparison between prostate cancer subgroups

| | | | CaP | CaP | |
|---|---|---|---|---|---|
| | Total | | negative | positive | |
| | (n = 191) | Range | (n = 141) | (n = 50) | P value |
| Mean age ± SEM (yrs) | 65.1 ± 0.6 | 41-90 | 64.8 ± 0.7 | 65.8 ± 1.5 | 0.508 |
| Mean prostate | 46.6 ± 2.1 | 6.9-210.8 | 48.5 ± 2.4 | 41.2 ± 4.3 | 0.131 |
| volume ± SEM (cc) | | | | | |
| Mean PSA ± SEM (ng/mL) | 7.86 ± 0.42 | 0.88-49.02 | 7.81 ± 0.51 | 7.98 ± 0.70 | 0.861 |
| Mean PSAD ± SEM | 0.202 ± 0.012 | 0.019-1.358 | 0.188 ± 0.013 | 0.242 ± 0.025 | 0.045 |
| Mean T-corrected | 0.00052 ± 0.00003 | 0.00005-0.0033 | 0.00047 ± 0.00003 | 0.00066 ± 0.00009 | 0.014 |
| PSAD ± SEM | | | | | |
| Mean cPSA ± SEM (ng/mL) | 6.00 ± 0.36 | 0.73-43.82 | 5.97 ± 0.44 | 6.09 ± 0.61 | 0.877 |
| Mean cPSAD ± SEM | 0.157 ± 0.010 | 0.013-1.209 | 0.146 ± 0.012 | 0.189 ± 0.022 | 0.067 |
| Mean T-corrected | 0.00041 ± 0.00003 | 0.00003-0.0030 | 0.00037 ± 0.00003 | 0.00052 ± 0.00008 | 0.022 |
| cPSAD ± SEM | | | | | |
| Mean testosterone ± | 429.6 ± 10.8 | 136-944 | 435.0 ± 12.8 | 414.6 ± 20.5 | 0.410 |
| SEM (ng/dL) | | | | | |

TABLE 3
Correlation of serum testosterone with age, prostate
volume, PSA, PSAD, T-corrected PSAD, cPSA, cPSAD,
T-corrected cPSAD and prostate cancer diagnosis

| | Total | | Hypogonadal | | Eugonadal | |
|---|---|---|---|---|---|---|
| | R value* | P value* | R value | P value | R value | P value |
| Age | −0.066 | 0.373 | 0.014 | 0.930 | −0.007 | 0.937 |
| Prostate | −0.209 | 0.004 | −0.100 | 0.540 | −0.146 | 0.073 |
| volume | | | | | | |
| PSA | 0.0002 | 0.998 | 0.094 | 0.565 | −0.007 | 0.933 |
| PSAD | 0.079 | 0.277 | 0.183 | 0.257 | 0.074 | 0.369 |
| T-cor- | −0.312 | <0.0001 | −0.015 | 0.925 | −0.197 | 0.015 |
| rected | | | | | | |
| PSAD | | | | | | |
| cPSA | 0.001 | 0.988 | 0.117 | 0.471 | 0.0004 | 0.997 |
| cPSAD | 0.064 | 0.377 | 0.181 | 0.265 | 0.071 | 0.385 |
| T-cor- | −0.287 | <0.0001 | 0.015 | 0.927 | −0.173 | 0.033 |
| rected | | | | | | |
| cPSAD | | | | | | |
| Prostate | 0.057 | 0.404 | 0.060 | 0.670 | 0.010 | 0.895 |
| cancer | | | | | | |
| diagnosis | | | | | | |

*R and P values refer to the bivariate analysis describing the correlation of testosterone with the dependent variables of age, prostate volume, PS A, PSAD, cPSA, cPSAD, and prostate cancer diagnosis.

What is claimed is:

1. A method of detecting prostate cancer in a subject, comprising the steps of:
a) collecting a sample of bodily fluid from a subject suspected of having prostate cancer;
b) determining the level of prostate specific antigen (PSA) and testosterone in the sample; and either:
i) determining the relationship between the PSA and the testosterone levels in the sample to obtain a testosterone corrected PSA; or
ii) measuring the prostate volume and relating the PSA level to the prostate volume and serum testosterone to obtain a testosterone corrected PSA density; and
c) comparing the testosterone corrected PSA or the testosterone corrected PSA density (PSAD) to a predetermined range of normal values;
wherein a subject having prostate cancer exhibits a testosterone corrected PSA level or a testosterone corrected PSA density outside the range of normal values.

2. The method of claim 1, wherein said subject is a human subject.

3. The method of claim 1, wherein said PSA is selected from the group consisting of free PSA, complexed PSA (cPSA), B-PSA, PRO-PSA and total PSA.

4. The method of claim 1, wherein said testosterone is selected from the group consisting of free testosterone, bioavailable testosterone, total testosterone and a testosterone-bound protein.

5. The method of claim 4, wherein the testosterone-bound protein is sex hormone binding globulin.

6. The method of claim 1, wherein said correcting for serum testosterone is achieved by dividing the serum PSA value by the product of the prostate volume times the serum testosterone value.

7. The method of claim 1, wherein said testosterone is determined by an immunoassay procedure.

8. The method of claim 1, wherein said PSA is determined by an immunoassay procedure.

9. The method of claim 6, wherein the prostate volume is determined by transrectal ultrasound (TRUS) measurements taken in the greatest dimension.

10. The method of claim 9, wherein said transrectal ultrasound measurements are calculated by the ellipsoid volume method of H×W×L×0.52.

11. A method of screening for, detecting or diagnosing prostate cancer in a subject comprising the steps of:
a. collecting a sample of bodily fluid from a subject suspected of having prostate cancer;
b. determining the level of testosterone and PSA present in said sample;
c. determining the prostate volume by ultrasound measurements; and
d. calculating the testosterone corrected PSA (Tc-PSA) and/or testosterone corrected PSA density (Tc-PSAD) and/or testosterone corrected PSA density of the transition zone (Tc-PSAD-TZ),
wherein a subject having prostate cancer has a testosterone corrected PSA (Tc-PSA) and/or testosterone corrected PSA density (Tc-PSAD) and/or testosterone corrected PSA density of the transition zone (Tc-PSAD-TZ) value which is significantly different from that obtained from a predetermined range of normal values established from screening normal non-cancerous individuals known to be free of prostate cancer.

12. The method of claim 11, wherein said subject is a human subject.

13. The method of claim 11, wherein said bodily fluid is selected from the group consisting of serum, plasma, whole blood, urine and saliva.

14. The method of claim 11, wherein said PSA is selected from the group consisting of free PSA, complexed PSA (cPSA), B-PSA, PRO-PSA, HK2 and total PSA.

15. The method of claim 11, wherein said testosterone is selected from the group consisting of free/bioavailable testosterone, total testosterone and a testosterone-bound protein.

16. The method of claim 15, wherein the testosterone-bound protein is sex hormone binding globulin (SHBG).

17. The method of claim 11, wherein said testosterone level is determined by an immunoassay procedure.

18. The method of claim 11, wherein said PSA level is determined by an immunoassay procedure.

19. The method of claim 11, wherein said calculating the testosterone corrected PSA (Tc-PSA) density, or Tc-PSAD-TZ of step d) is accomplished by dividing the PSA value in the bodily fluid sample by the product of the prostate volume times the testosterone value in the bodily fluid sample.

20. The method of claim 19, wherein said prostate volume is based on transrectal ultrasound (TRUS) measurements taken in the greatest dimension.

21. The method of claim 20, wherein said transrectal ultrasound (TRUS) measurements are calculated by the ellipsoid volume method of H×W×L×0.52.

22. A method of identifying a subject at risk for developing prostate cancer, said method comprising the steps of:
a. collecting a sample of bodily fluid from a subject suspected of being at risk for developing prostate cancer;
b. determining the level of testosterone and PSA present in said sample;
c. determining the prostate volume by ultrasound measurements; and
d. calculating the testosterone corrected PSA density (Tc-PSAD) or testosterone corrected PSA density of the transition zone (Tc-PSAD-TZ),
wherein a subject suspected of being at risk for developing prostate cancer has a Tc-PSA value or a Tc-PSA-TZ value which is significantly different from that obtained from a predetermined range of normal values established from screening normal non-cancerous individuals known to be free of prostate cancer.

23. The method of claim 22, wherein said sample of bodily fluid is collected at least twice a year from said subject and the Tc-PSA level changes in value over time compared to baseline levels, and wherein said Tc-PSA levels fall significantly outside of the range of levels observed in subjects free of prostate cancer.

24. The method of claim 22, wherein said subject is a human subject.

25. The method of claim 22, wherein said PSA is selected from the group consisting of free PSA, complexed PSA (cPSA), B-PSA, PRO-PSA and total PSA.

26. The method of claim 22, wherein said testosterone is selected from the group consisting of free or bioavailable testosterone, total testosterone and a testosterone-bound protein.

27. The method of claim 26, wherein the testosterone bound protein is sex hormone binding globulin (SHBG).

28. The method of claim 22, wherein said testosterone level is determined by an immunoassay procedure.

29. The method of claim 22, wherein said PSA level is determined by an immunoassay procedure.

30. The method of claim 22, wherein said calculating the testosterone corrected PSA (Tc-PSA) density or testosterone corrected PSA density of the transition zone (Tc-PSAD-TZ) of step d) is accomplished by dividing the PSA value from the sample of bodily fluid by the product of the prostate volume times the testosterone value obtained from said sample of bodily fluid.

31. The method of claim 30, wherein said prostate volume is based on transrectal ultrasound (TRUS) measurements taken in the greatest dimension.

32. The method of claim 31, wherein said transrectal ultrasound (TRUS) measurements are calculated by the ellipsoid volume method of H×W×L×0.52.

33. A method for pre-treatment staging of prostate cancers in a subject having prostate cancer, said method comprising the steps of:
a) collecting a serum sample from a subject having prostate cancer;
b) determining the level of testosterone and PSA present in said serum sample;
c) determining prostate volume by ultrasound measurement; and
d) calculating the testosterone corrected PSA (Tc-PSA) density or testosterone corrected PSA density of the transition zone (Tc-PSAD-TZ),
wherein the testosterone corrected PSA (Tc-PSA) density or testosterone corrected PSA density of the transition zone (Tc-PSAD-TZ) is calculated by dividing the PSA value by the product of the prostate volume times the testosterone value.

34. The method of claim 33, wherein said subject is a human subject.

35. The method of claim 33, wherein said PSA is selected from the group consisting of free PSA, complexed PSA (cPSA), B-PSA, Pro-PSA and total PSA.

36. The method of claim 33, wherein said testosterone is selected from the group consisting of free/bioavailable testosterone, total testosterone and a testosterone-bound protein.

37. The method of claim 36, wherein the testosterone bound protein is sex hormone binding globulin (SHBG).

38. The method of claim 33, wherein said testosterone level is determined by an immunoassay procedure.

39. The method of claim 33, wherein said PSA level is determined by an immunoassay procedure.

40. The method of claim 33, wherein said calculating the testosterone corrected PSA (Tc-PSA) density and testosterone corrected PSA density of the transition zone (Tc-PSAD-TZ) of step d) is accomplished by dividing the PSA value from the sample of bodily fluid by the product of the prostate volume times the testosterone value obtained from the sample of bodily fluid.

41. The method of claim 40, wherein said prostate volume is based on transrectal ultrasound (TRUS) measurements taken in the greatest dimension.

42. The method of claim 41, wherein said transrectal ultrasound (TRUS) measurements are calculated by the ellipsoid volume method of H×W×L×0.52.

43. The method of any one of claims 11, 22 or 33, wherein said method demonstrates a negative correlation between prostate volume and testosterone levels.

44. The method of any one of claims 11, 22 or 33, wherein said sample of bodily fluid is selected from whole blood, blood cells, plasma, serum, urine and saliva.

45. A kit for measuring one or more isoforms of PSA and one or more forms of testosterone, wherein the PSA isoform is selected from the group consisting of free PSA, cPSA, Pro-PSA, B-PSA, HK2 and/or total PSA and the forms of testosterone are selected from the group consisting of free or bioavailable testosterone, total testosterone and any testosterone-bound protein such as sex hormone binding globulin (SHBG) for the purpose of testosterone correction of PSA, in a subject comprising:
a. a solid substrate comprising an immobilized binding partner specific for at least one or more PSA isoforms and at least one or more forms of testosterone or testosterone bound protein; and
b. either:
i) an enzyme conjugated second binding partner capable of binding to the PSA isoform of step a) and testosterone; or
ii) a biotinylated second binding partner capable of binding to the PSA isoform of step a) and testosterone; and
c. either:
i) an enzyme substrate and a developing reagent specific for the enzyme conjugated second binding partner of step b) i); or
ii) a streptavidin conjugated third binding partner specific for the second binding partner of step b) ii); and
d. a buffer for washing and sample dilution; and
e. a standard for a PSA isoform and testosterone or testosterone bound protein; and
f. instructions for using the kit.

46. The kit of claim 45, wherein said PSA is selected from the group consisting of free PSA, complexed PSA, B-PSA, PRO-PSA and total PSA.

47. The kit of claim 45, wherein said testosterone is selected from the group consisting of free or bioavailable testosterone, total testosterone and any testosterone-bound protein such as sex hormone binding globulin (SHBG) for the purpose of testosterone correction of PSA.
